(19) United States
(12) Patent Application Publication
(10) Pub. No.: US 2009/0175845 A1
(43) Pub. Date: Jul. 9, 2009

(54) METHODS FOR IDENTIFYING COMPOUNDS THAT MODULATE PHB DOMAIN PROTEIN ACTIVITY AND COMPOSITIONS THEREOF

(75) Inventors: Martin Chalfie, New York, NY (US); Thomas Benzing, Cologne (DE)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, NEW YORK, NY (US)

(21) Appl. No.: 12/091,223

(22) PCT Filed: Oct. 24, 2006
(86) PCT No.: PCT/US2006/041379

§ 371 Date: Oct. 28, 2008

Related U.S. Application Data
(60) Provisional application No. 60/729,974, filed on Oct. 25, 2005.

(51) Int. Cl. A61K 31/7088; C12Q 1/68; C07H 21/04; A61K 39/395; A61K 31/56; A61P 43/00

(52) U.S. Cl. 424/130.1; 435/6; 536/23.5; 514/44; 514/182

(57) ABSTRACT

The invention relates to the finding that PHB domain polypeptides can bind cholesterol and related compounds and are involved in the regulation of various cellular activities. Accordingly, the invention includes methods of identifying compounds that can bind to and/or modulate activity of a PHB domain polypeptide such as Podocin or MEC2.

This application claims priority to provisional U.S. Application Ser. No. 60/729,974, filed on Oct. 25, 2005, which is herein incorporated by reference in its entirety.

The U.S. Government may have certain rights in this invention pursuant to Grant No. GM30997 awarded by the National Institutes of Health.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

Prohibitin homology (PHB) domain proteins are conserved membrane-associated proteins that regulate osmotic homeostasis, mechanosensation, and signaling. Several PHB domain proteins are associated with membrane microdomains (sometimes referred to as lipid rafts) in the plasma membrane.

The PHB domain protein MEC-2 is part of a multiprotein channel complex that includes at least the PHB-domain protein UNC-24, the DEG/ENaC (degenerin/epithelial Na+ channel) proteins MEC-4 and MEC-10 and the paraoxonase-like protein MEC-6 that transduces gentle touch. Mutations in Podocin, a PHB domain protein of the mammalian kidney, result in focal segmental glomerulosclerosis, a severe genetic kidney disorder in humans. Podocin specifically localizes to the slit diaphragm, a specialized cell contact that is part of the glomerular filtration barrier of the kidney. Slit diaphragm proteins induce signal transduction in podocytes, the visceral epithelial cells of the kidney glomerulus, which regulate cytoskeletal rearrangement and transcriptional activity.

### SUMMARY OF THE INVENTION

The invention is related, in part, to the findings provided herein that using two PHB domain proteins: MEC-2, which is part of the channel complex that transduces gentle touch, and Podocin, which is needed for the function of the filtration barrier in the human kidney. The invention relates to the finding that rather than being recruited to preexisting lipid domains, these proteins directly interact with cholesterol and recruit it into membrane protein complexes. This is achieved by the combination of direct lipid interaction and multimerization by protein-protein interactions. Both proteins interact with and regulate the activity of ion channels: MEC-2 with the DEG/ENaC channel protein MEC-4 and, as shown herein, Podocin with the TRPC6 canonical transient receptor potential channel protein. Consistent with a critical role for Podocin in regulating the sterol surrounding of TRPC channels, limited cholesterol depletion results in the loss of Podocin-mediated augmentation of TRP channel conductance and mutant Podocin deficient in cholesterol binding fails to facilitate TRPC channel activation. These data show that MEC-2 and Podocin similarly regulate the lipid microenvironment of ion channels, assign a novel role for membrane sterols in participating in the formation of membrane protein supercomplexes, and suggest that cholesterol plays an important role in regulating ion channel activity.

In one aspect, the invention provides a method for regulating the lipid microenvironment of ion channels, the method comprising inhibiting or enhancing an interaction between a PHB domain protein and a membrane sterol. The membrane sterol can be, for example, cholesterol. In all aspects of the invention, a PHB domain protein includes, but is not limited to, MEC-2 and Podocin. In all aspects of the invention, inhibiting an interaction between a PHB domain protein and a membrane sterol or a membrane-associated protein can be performed by using a small-molecule, a protein, a peptide, or some other agent that can either bind to the PHB domain protein, the sterol, or the membrane-associated protein such that interactions are prevented or enhanced.

In one aspect, the invention provides a method for regulating the lipid microenvironment of ion channels, the method comprising inhibiting or enhancing an interaction between a PHB domain protein and a membrane-associated protein. The membrane-associated protein can be, for example, a membrane channel protein. A membrane channel protein can be, for example, an ion membrane channel protein.

In one aspect, the invention provides a method for regulating the activity of ion channels, the method comprising inhibiting or enhancing an interaction between MEC-2 with MEC-4.

In one aspect, the invention provides a method for regulating the activity of ion channels, the method comprising inhibiting or enhancing an interaction between Podocin with TRPC6.

In one aspect, the invention provides a method for blocking TRPC channel activation, the method comprising inhibiting an interaction between Podocin and a sterol or between Podocin and TRPC6.

In one aspect, the invention provides a method for blocking TRPC channel activation, the method comprising introducing into a cell a mutant Podocin that cannot bind to cholesterol. The introduction can be, for example, by transfection (stable or transient) of the cell with an expression vector that produces or overexpresses the mutant Podocin. The introduction can also be conducted by other means, such as by liposome or by infection with a viral vector or virus particle.

In another aspect, the invention provides methods for screening for compounds or agents that can inhibit the interactions between PHB domains and sterols and membrane-associated proteins as described herein. In one aspect, the methods for screening for compounds or agents are directed to compounds or agents that can inhibit the interactions between MEC-2 or Podocin with sterols or membrane-associated proteins. The sterol can be, for example, cholesterol. The membrane-associated proteins can be, for example, ion channel membrane proteins. Exemplary interactions can be, for example, be MEC-2 with the DEG/ENaC channel protein MEC-4 and, Podocin with the TRPC6. The screening methods can be, for example, in accordance with the protocols described herein.

Additional aspects of the invention include a method for identifying a compound that modulates a PHB domain protein for which the method includes providing a polypeptide containing a PHB domain, contacting the polypeptide with a compound, and determining whether or not the compound binds to the polypeptide, such that binding of the compound to the polypeptide indicates that the compound modulates the PHB domain protein. In some embodiments, the polypeptide containing a PHB domain is located within a membrane. The method can further include detecting whether there has been an increase or a decrease in PHB domain polypeptide activity. The detecting can include measuring cholesterol or other sterol bound by the PHB domain, measuring protein bound by the PHB domain polypeptide, measuring multimerization of the PHB polypeptides, measuring ion channel activity, measuring cholesterol or sterol recruitment, measuring enzymatic activity, or any combination thereof. In certain cases, the PHB domain polypeptide includes a peptide consisting of a PHB domain, a peptide having a hydrophobic domain linked to a PHB domain, a peptide having a PHB domain linked to about five additional amino acids, or any of the forgoing having a mutation in a palmitoylation site. In one example, the PHB domain polypeptide is the PHB domain of the amino acid sequence corresponding to GenBank Accession No. AY050309. The PHB domain polypeptide can, in some embodiments, include the consecutive amino acids from 124 to 285 of a murine Podocin amino acid sequence (e.g., of SEQ ID NO:1), the consecutive amino acids from 138 to 300 of a C. elegans MEC-2 amino acid sequence (e.g., of SEQ ID NO:2), or a homolog or ortholog thereof. The compound of the method can be, e.g., a cholesterol, a sterol, a steroid, a phosphatidyl ethanolamine, or an analog of any thereof. In certain embodiments, the method further includes providing a known ligand of the PHB domain polypeptide and determining the relative binding of the test compound compared to the known ligand. In some cases, the PHB domain polypeptide is expressed in a tissue and the detecting is performed in the tissue sample. The PHB domain polypeptide is, in certain embodiments, expressed in a cell and the detecting is performed by assaying the amount of test compound in the low density fraction (LDF) fraction derived from the cell, wherein a decrease in the amount of PHB domain polypeptide in the LDF fraction compared to a control indicates that the test compound can bind to the PHB domain polypeptide. In yet other embodiments, the detecting includes determining the amount of compound bound to the PHB domain polypeptide.

The invention also relates to a method for identifying a compound that modulates a PHB domain protein for which the method includes (a) providing a protein containing a PHB domain, and a target protein, such that the target protein is capable of being bound by the PHB domain protein, (b) contacting the proteins of (a) with a compound, and (c) determining whether the compound inhibits or enhances binding of the PHB domain protein with the target protein. In some embodiments of the method, the PHB domain protein and target protein are admixed with membrane lipids. In certain cases, the method further comprises determining the affinity of the compound for the PHB domain polypeptide. The PHB domain protein can include Podocin and the target protein can include TRPC6. The PHB domain protein can, in certain embodiments, include stomatin and the target protein includes TRPC1. In yet other embodiments of the method, the PHB domain protein includes flotillin and the target protein includes an Alzheimer's precursor protein (APP). In some cases, the PHB domain protein comprises a pannexin, the PHB domain protein contains a mutation (e.g., the PHB domain protein contains a mutation in a palmitoylation site).

In one aspect, the invention relates to a method for identifying a compound that modulates activity of a PHB domain polypeptide for which the method includes (a) providing a PHB domain polypeptide; (b) contacting the PHB domain polypeptide with a compound under conditions suitable for detecting PHB domain protein activity; and (c) detecting PHB domain protein activity and comparing the activity detected with activity detected from a PHB domain polypeptide in the absence of the compound, so as to identify a compound that modulates PHB domain polypeptide activity. In some aspects, the PHB domain polypeptide activity detected is cholesterol or sterol binding. The PHB domain polypeptide activity detected can be ion channel activity. In certain aspects, the PHB domain polypeptide is the amino acid sequence corresponding to GenBank Accession No. AY050309 and ion channel activity of a TRPC ion channel is detected. In certain aspects, the PHB domain polypeptide is a PHB domain protein. The PHB domain polypeptide can be a polypeptide having the amino acid sequence corresponding to GenBank Accession No. AY050309 or an ortholog thereof. In another aspect, the PHB domain polypeptide comprises (i) the consecutive amino acids from 124 to 285 of a murine Podocin amino acid sequence, (ii) the consecutive amino acids from 138 to 300 of a C. elegans MEC-2 amino acid sequence, or (iii) a homolog or ortholog thereof. The compound of the method can be, e.g., a cholesterol, a sterol, a steroid, a phosphatidyl ethanolamine, or an analog of any thereof. In some cases, the PHB domain polypeptide is in an intact organism or tissue from an organism, e.g., a C. elegans. In yet another aspect, the assay uses a C. elegans, and the assay is a touch sensitivity assay, wherein a test compound that binds to MEC-2, interferes with binding of MEC-2 to cholesterol, interferes with binding of MEC-2 to other PHB domain proteins, interferes with the binding of MEC-2 to MEC-2, to cholesterol or to themselves or other PHB-domain proteins modulates touch sensitivity in a wild-type C. elegans by decreasing touch sensitivity, or in a mutant for touch sensitivity by increasing touch sensitivity. In another aspect, the organism is a C. elegans, and the assay is a touch sensitivity assay, such that a test compound that binds to a PHB domain protein interferes with the binding of a PHB domain protein to cholesterol, interferes with the binding of the PHB domain protein to itself, interferes with the binding of the PHB domain protein to a different PHB domain protein modulates touch sensitivity in a wild-type C. elegans by decreasing touch sensitivity, or in a mutant for touch sensitivity, by increasing touch sensitivity. In other aspects, the detecting comprises detecting ion channel activity, e.g., the detecting of ion channel activity is performed using a X. laevis oocyte system. The detected ion channel activity is, in some cases, at least one of Na⁺ channel activity, Ca²⁺ channel activity, K⁺ channel activity, or non-selective cation channel activity. In certain aspects of the method the detecting comprises detecting membrane receptor activity or membrane protein activity, e.g., a G-protein coupled membrane receptor activity or a hormone receptor activity. In yet other aspects of the method, the compound can bind to a pannexin and can modulate lysis in a red blood cell. In certain aspects, the PBH domain polypeptide is a mutated PHB domain polypeptide, e.g., the PHB domain polypeptide has a mutation in at least one predicted palmitoylation site.

In another embodiment, the invention relates to a method of identifying a compound that can modulate multimerization of a PHB domain polypeptide. The method includes (a) providing a population of PHB domain polypeptides; (b) contacting the population with a compound; and (c) determining whether the PHB domain proteins form multimers, such that an inhibition of multimerization in the population of (b) as compared to a population in the absence of a compound, indicates that the compound can modulate multimerization of a PHB domain polypeptide. In some cases, the compound is a cholesterol, sterol, steroid, phosphatidylethanolamine or a compound related to any of the forgoing.

The invention also relates to an isolated peptide consisting essentially of a PHB domain. In one embodiment, the invention includes an isolated peptide consisting essentially of a PHB domain linked to 5 additional amino acids, e.g., linked to the amino terminus of the PHB domain. In certain aspects, the isolated peptide has the amino acid sequence of amino acids 124-285 of SEQ ID NO:1 or amino acids 138-300 of SEQ ID NO:2. The invention also relates to a composition comprising any of the peptides disclosed herein and a pharmaceutically acceptable carrier. In certain aspects, a composition is one such that the PHB domain is a MEC-2 PHB domain, a Podocin PHB domain, an MEC-2 PHB domain having 5 additional amino acids at the amino terminus, or a Podocin PHB domain having 5 additional amino acids at the amino terminus.

In another embodiment, the invention relates to a method for modulating activity of a PHB domain polypeptide. The method includes contacting a cell expressing the PHB domain polypeptide with an agent that can bind to the PHB domain of the PHB domain polypeptide. In certain aspects, the agent is an antibody that specifically binds a PHB domain protein. In some cases, the agent is a peptide consisting essentially of a PHB domain. In another aspect, the agent is a cholesterol, a sterol, or a steroid, e.g., lathosterol, ergosterol, or 7-dehydro cholesterol.

### BRIEF DESCRIPTION OF THE DRAWINGS

FIG. 1A is a photographic reproduction of an immunoblots depicting immunoprecipitation of FLAG-tagged Podocin (F.Podocin), FLAG-tagged MEC-2 (F.MEC-2), sIg.7-tagged MEC-2, and a control protein (sIg.7).

FIG. 1B is a photographic reproduction depicting the results of velocity gradient centrifugation after mild detergent solubilization. Fractions were collected from the top (fraction 1) to the bottom of the tube (fraction 8). Faster migration indicates multimerization.

FIG. 1C is a photographic reproduction of the results of a Western blot of density gradient centrifugation products. Fractions of the lysates after floatation gradient ultracentrifugation obtained from the top (fraction 1) to the bottom (fraction 7) of the tube were analyzed by Western blot with the respective antibodies. Detergent-resistant light fractions (DRM; low density fraction (LDF)), non-detergent-resistant membrane domains (NDRM), Transferrin receptor (TFR).

FIG. 1D is a photographic reproduction of an immunoblots depicting the results of experiments conducted as for 1C, in which samples were cholesterol depleted with MBCD or treated with MBCD and cholesterol (MBCD+Chol).

FIG. 1E is a bar graph depicting experiments quantifying the cholesterol content in DRM fractions. The overall cholesterol content was identical in all conditions.

FIG. 2A is a reproduction of an autoradiogram demonstrating the results of experiments in which MEC-2 and Podocin expressed in HEK 293T cells was incubated with photoactivatable [³H]photocholesterol (PA-CHOL) and photoactivatable [³H]phosphatidylcholine (PA-PC) for 16 hours. Photolabeled proteins were resolved by SDS-PAGE and visualized by autoradiography (upper panels). Lower panels depict expression of proteins in the lysates.

FIG. 2B is a photographic reproduction of the results of an immunoprecipitation experiment.

FIG. 2C is a photographic reproduction of a binding experiment in which a fusion protein with a carboxy terminal fusion of the PHB-domain and the hydrophobic region of Podocin (amino acids 105-284) with the extracellular and transmembrane domains of Nephrin binds photoactivatable cholesterol indicating that this domain can convey cholesterol binding.

FIG. 2D is a bar graph depicting the results of a binding experiment testing direct binding of cholesterol to Podocin. NusA or NusA fused to the cholesterol binding domain of Podocin (amino acids 119-284) was incubated with radioactively labeled cholesterol, washed extensively and subjected to scintillation counting.

FIG. 3 is a reproduction of an immunoblots depicting the results of experiments in which digitonin-precipitated cell extracts were probed for nephrin and Podocin. The left panel is an immunoblots of the 15,000×g pellet after additional of digitonin (+) or solvent (−). The right panel is an immunoblots of the input lysates.

FIG. 4 is a bar graph depicting the results of experiments in which NusA or NusA fused to the cholesterol binding domain of Podocin (amino acids 119-124) was incubated with radioactive cholesterol in the presence of absence of unlabeled cholesterol.

FIG. 5A is a reproduction of a photoimage of native gel electrophoresis of cellular lysates. The positions of FLAG-tagged Podocin (F. Podocin^{WT}; #) and monomeric (*) and dimeric (**) F. Podocin^{ΔPHB} are indicated.

FIG. 5B is a reproduction of a gel showing the results of experiments in which cell lysates were subjected to velocity gradient centrifugation after mild detergent solubilization and Flag-tagged Podocin and a Flag-tagged Podocin mutant were detected. The position of multimeric Podocin is indicated. MBCD is methyl-β-cyclodextrin.

FIG. 6A is a drawing depicting the structure and membrane orientation of Podocin and MEC-2. The hydrophobic region (light grey) inserts into the inner leaflet of the plasma membrane causing the remaining parts of the protein, including the PHB-domain (dark grey) to face the cytoplasm and the inner leaflet. Sites of palmitate attachment are indicated by wavy lines.

FIG. 6B is a representation of the alignment of the predicted sequence of mouse Podocin and C. elegans MEC-2 showing the hydrophobic region (light grey), PHB-domain (dark grey), palmitoylation sites (open triangles), and the site of the proline to serine mutation in the hydrophobic region that prevents cholesterol binding (filled triangle).

FIG. 7A is a set of photographic reproductions of the results of experiments in which photolabeled proteins were immunoprecipitated with anti-FLAG antibody, resolved by SDS-PAGE and visualized by autoradiography (upper panel). The lower panel shows the expression of proteins in the lysates.

FIG. 7B is a photographic reproduction of the results of velocity gradient centrifugation to determine whether wild type and MEC-2(P134S) multimerize.

FIG. 7C is a photographic reproduction of the results of immunoprecipitation experiments testing coimmunoprecipitation of Wild-type MEC-2 and MEC-2(P134S) with V5-tagged rat αENaC from HEK 293T cells (the second transmembrane domain of αENaC can substitute for that of MEC-4 in vivo [Hong et al. (1994) Nature 367:470-3]. The rat channel protein was used because of an inability to express MEC-4 to sufficient levels in this system.

FIG. 7D is a photographic reproduction depicting the results of an experiment localizing wild type and mutant MEC-2 in processes of touch receptor neurons in C. elegans. Proteins were visualized in C. elegans with a MEC-2-specific antibody Zhang et al. (2004) Curr. Biol. 14:1888-96].

FIG. 8A is a photographic reproduction of an autoradiogram depicting the results of palmitoylation experiments in which alanine was substituted for cysteine in two predicted palmitoylation sites.

FIG. 8B is a photographic reproduction of an autoradiogram depicting the results of cholesterol binding experiments in which alanine was substituted for cysteine in two predicted palmitoylation sites.

FIG. 8C is a bar graph depicting the results of experiments testing touch sensitivity in MEC-2(C140/174A) mutants (black bars) requires cholesterol or its derivatives. Responses of wild-type animals (white bars) are also shown and not affected by limited cholesterol depletion. mec-2 null worms were transformed with the mec-2(C140/174A) gene and grown on plates with defined sterol concentrations prior to analysis of touch sensitivity. Depicted is the mean+/−SEM (number of animals tested is indicated; **p<0.001 as compared to mec-2(C40/174A) at high cholesterol).

FIG. 8D is a bar graph depicting the results of experiments testing the effect of severe cholesterol depletion on the sensitivity of wild type animals (white bars) and mec-2(C140/174A) mutants (black bars). Worms grown on plates containing 20 nM cholesterol for three generations were either maintained on 20 nM cholesterol for another generation or placed on cholesterol-free plates prior to analysis of response to gentle touch (number of animals tested is indicated; **p<0.001 as compared to wild-type worms on 13 μM cholesterol, # p<0.001 as compared to mutants on 13 μM cholesterol).

FIG. 9A is a pair of photographic reproduction depicting the results of experiments in demonstrating that mouse TRPC6 co-immunoprecipitates with FLAG-tagged Podocin (F.Podocin) but not with a control protein (F.GFP). Upper panel shows coprecipitated TRPC6 channel after immunoprecipitation of Podocin or GFP. Middle and lower panels show the expression of the proteins in the lysates.

FIG. 9B is a reproduction of a photomicrograph and an interpretive drawing of the photomicrograph demonstrating that TRPC6 is located at the slit diaphragm (SD) of podocyte foot processes (FP) near the glomerular basement membrane (GBM). This localization mimics that of Podocin. Arrows indicate the localization of gold particles in the electron micrograph.

FIG. 10A is a reproduction of an image of a set of PCR products produced using primer pairs specific for the individual TRPC channels as indicated.

FIG. 10B is a reproduction of an immunoblots of TRPC channels bound to FLAG-tagged Podocin (F.Podocin^{WT}) or a control protein (F.GFP), that were co-expressed with HA-tagged TRPC channels (mouse TRPC1 to TRPC6) and detected with an anti-HA antibody.

FIG. 11A is a set of graphs depicting the results of experiments testing TRPC6 currents in Xenopus oocytes stimulated with 10 μM 1-oleoyl-2-acetyl-sn-glycerol (OAG).

FIG. 11B is a bar graph depicting the results of experiments testing whether Podocin increases the effect of OAG (10 μM 1-oleoyl-2-acetyl-sn-glycerol; black bars) on NMDG-sensitive conductance (G_{NMDG}) of TRPC6 channels in Xenopus oocytes compared to but mutant Podocins. Currents in control oocytes (white bars) were not affected. The Podocin mutants used were Podocin^{ΔPHB}, Podocin^{P120S} and Podocin^{C126/160A} (see FIGS. 6A and 6B). The number of oocytes examined is given in parentheses. *p<0.05 as compared to water-injected oocytes; # p<0.05 as compared to TRPC6 coexpressed with Podocin^{ΔPHB}.

FIG. 11C is a graph depicting the results of experiments comparing the ability of wild type and mutant, Podocin to increase histamine-induced calcium influx (measured as a changed in fluorescence, ΔF/F) in HeLa Cx43 cells. Cells were transiently mock transfected () or transfected with DNA coding for wild-type Podocin (▴), Podocin^{ΔPHB} (Δ), Podocin^{C126/160A} (▾), and measured simultaneously in the same experiment using a FLIPR. Data are means±SD from 3-5 independent experiments. Measurements are taken at the shoulder of the response (line in the inset, which shows the calcium responses of the cells challenged with 10 μM histamine). Vertical scale, 10⁴ arbitrary fluorescence units; horizontal scale, two minutes.

FIG. 11D is a graph depicting the results of experiments conducted as for FIG. 11C except that cells were treated with MBCD to deplete cholesterol.

FIG. 12A is a bar graph depicting the results of experiments in which a cholesterol-binding domain (PHB domain) of Podocin (amino acids 119-184) or amino acids 1-99 of Podocin were tested for binding to cholesterol.

FIG. 12B is a bar graph depicting the results of experiments in which the PHB domain of Podocin was tested for binding to dexamethasone.

### DETAILED DESCRIPTION OF THE INVENTION

It has been found that PHB-domain proteins bind and recruit sterols to influence the activity of associated proteins in membrane protein supercomplexes. Because many of the PHB-domain proteins participate in protein complexes involved in important human disease (nephropathy, hypertension, Alzheimer's, immunological disorders) this finding that PHB-domain proteins recruit sterols is important for drug development. For example, high-throughput assays based on modulating the interaction between PHB-domain proteins and sterols are useful for identifying compounds that can be used as drugs to modulate this interaction.

In addition, thus far little is known about the role of lipid binding for the regulation of ion channels. Compounds that modulate the interaction between PHB domains that are associated with ion channels and sterols are useful for identifying drugs for modulating channel activity, e.g., for regulating nervous system activity.

In addition to the well-know effects of steroids that are mediated by binding to cytosolic and/or nuclear receptors, PHB-domain proteins may mediate some of the non-transcriptional effects of steroids. This is confirmed by data demonstrating that a PHB domain protein can not only bind cholesterol or sterols of the membrane but can also interact with glucocorticoids (dexamethasone).

The invention is based, in part, on the finding that Podocin and MEC-2 are cholesterol binding proteins and that cholesterol binding plays an important role in regulating the activity of ion channels to which these PHB-domain proteins bind. Podocin, as demonstrated herein, binds to, colocalizes at the slit diaphragm with, and regulates the activity of TRPC6. It is also shown that MEC-2 binds to DEG/ENaC channels. These findings indicate that these proteins, similar to other proteins associated with MEC-2, are part of a mechanosensitive protein complex at the slit diaphragm of podocytes. Based on these and other data, many of the PHB-domain proteins regulate membrane protein function by binding sterols, e.g., by altering their local lipid environment.

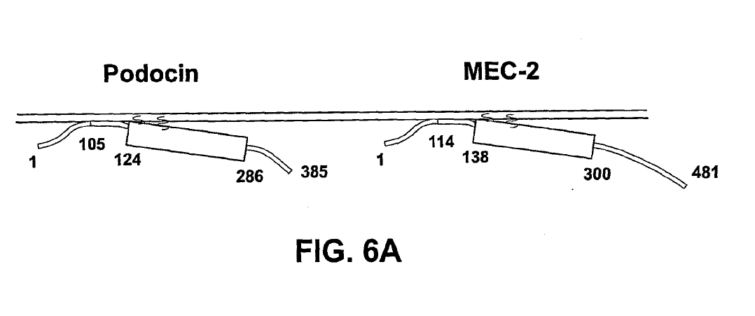

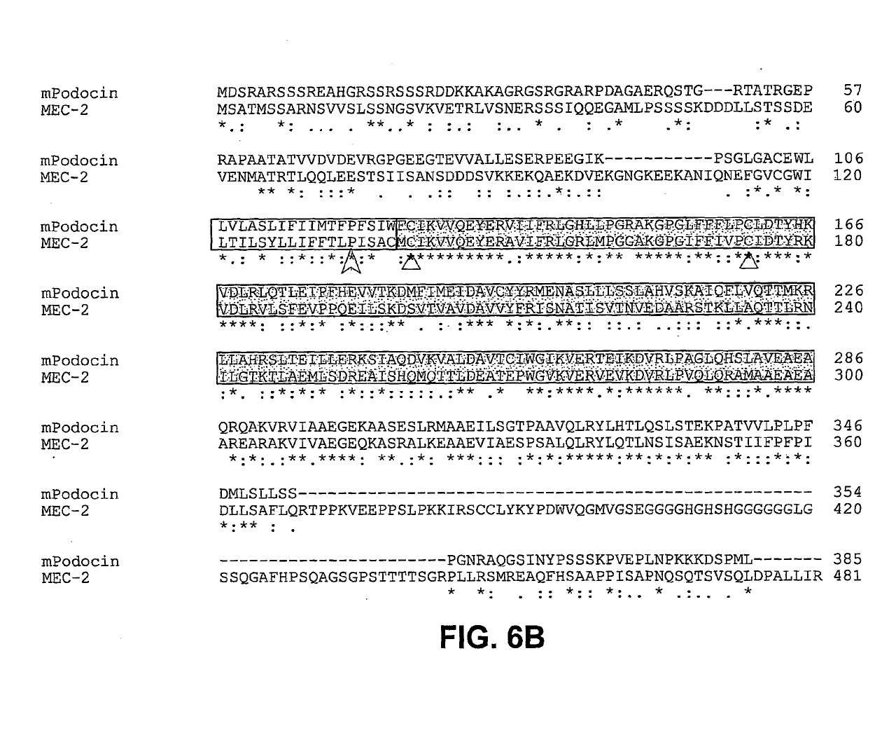

MEC-2 and Podocin are predicted to form hairpin-like structures with a single, central hydrophobic domain close to the plasma membrane and amino- and carboxy-terminal tails facing the cytoplasm (FIG. 6A). Although the two proteins contain different N- and C-termini, they have PHB domains that are 50% identical and 80% similar. The PHB domain, which is C-terminal to the hydrophobic region of Podocin and MEC-2 (FIG. 6B, dark shaded box), is critical for the action of both proteins. The function of this conserved domain is unclear.

### Screening Assays

The invention provides methods (also referred to herein as “screening assays”) for identifying modulators, i.e., test compounds (e.g., small non-nucleic acid organic molecules, small inorganic molecules, proteins, peptides, peptidomimetics, peptoids, heteroorganic molecules, organometallic molecules, or other drugs) that bind to a PHB domain polypeptide. As used herein, a “PHB domain polypeptide” refers to a protein that includes a PHB domain protein or a fragment of a PHB domain protein (i.e., a PHB domain peptide) that retains the ability to bind a ligand of the PHB domain protein (e.g., cholesterol or related molecule), have a stimulatory or inhibitory effect on, for example, a PHB domain protein activity, or have a stimulatory or inhibitory effect on, for example, the activity of a protein that interacts with a PHB domain protein. Compounds thus identified can be used to modulate the activity or effects of a PHB domain protein, for example, in a therapeutic protocol, to elaborate the biological function of the PHB domain protein, or to identify compounds that disrupt normal PHB domain protein interactions.

In one embodiment, the invention provides assays for screening candidate or test compounds that can bind to a PHB domain protein or polypeptide. In another embodiment, the invention provides assays for screening candidate or test compounds that modulate the activity of a PHB domain protein or polypeptide.

### PHB Domain Polypeptides

PHB domain polypeptides are known in the art and include known proteins containing PHB domains and homologs and orthologs of such proteins. Also included are PHB domains derived from PHB domain proteins. Examples of PHB domain proteins include, without limitation, prohibitin, Podocin, stomatin, flotillin, MEC-2, vacuolin A, vacuolin B, UNC-1, UNC-24 and orthologs of the forgoing. Specific addition proteins having PHB domains are known in the art and those of skill in the art can identify a PHB domain [Morrow and Parton, (2005) Traffic 6, 725-740].

Non-limiting examples of PHB domain proteins include, without limitation, the amino acid sequences corresponding to GenBank Accession Nos. AY050309 (murine Podocin), U26735 (MEC-2), NM-008027 (murine Flotillin-1), NM_{—}004475 (murine Flotillin-2), NM_{—}019482 (murine Pannexin-1), NM_{—}001002005 (murine Pannexin-2), NM_{—}172454 (murine Pannexin-3), and orthologs thereof (e.g., human orthologs). Additional examples of PHB domain containing polypeptides include SEQ ID NO:1, amino acids 124-286 of SEQ ID NO:1, amino acids 124-285 of SEQ ID NO:1, SEQ ID NO:2, amino acids 139-300 of SEQ ID NO:2, amino acids 125-286 of SEQ ID NO:1, amino acids 120-286 of SEQ ID NO:1, and amino acids 124-300 of SEQ ID NO:2. In one embodiment, the PHB domain protein can be a polypeptide having from about 75% identity to about 99% identity to the PHB domain sequences shown in SEQ ID NOS: 1 or 2. In one embodiment, the PHB domain protein can be a polypeptide that is about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76% or about 75% identical to the PHB domain sequence shown in SEQ ID NO: 1 or 2 or the PHB domain sequences shown in GenBank Accession Nos. AY050309 (murine Podocin), U26735 (MEC-2), NM-008027 (murine Flotillin-1), NM_{—}004475 (murine Flotillin-2), NM_{—}019482 (murine Pannexin-1), NM_{—}001002005 (murine Pannexin-2), NM_{—}172454 (murine Pannexin-3).

### Compounds

Test compounds are generally compounds that are sterols, cholesterol, cholesterol analogs, cholesterol derivatives, steroids, or other compounds with properties similar to such molecules. Such compounds include animal and plant sterols and steroids including phytoestrogens, cholesterol analogs including without limitation epicholesterol, lathosterol, dihydrocholesterol, ergosterol, desmosterol, 25-hydroxycholesterol, lanosterol, androstenolone, coprostanol, cholestane, 7-dehydro cholesterol, and cholestenone) and compounds related to steroids such as calciferol and cholecalciferol. Additional useful compounds include phosphatidylethanolamine and related compounds. In some cases useful compounds are derivatized or labeled so they can be used in a particular assay. For example a head group modified, photoreactive analog of phosphatidylethanolamine, N-[¹²⁵I]iodo-4-azidosalicylamidyl)-1,2-dilauryl-sn-glycero-3-phosphatidylethanolamine is useful for photoaffinity labeling studies, e.g., in RBCs, particularly for binding to stomatin. Another example of such a compound is 4-hydroxy-3-iodo [¹²⁵I])-N-[2-(2-pyridinyldithio)ethyl]-benzenepropanamide [Desneves et al. (1996) Biochem. Biophys. Res. Comm. 224:108-114].

Useful compounds also include those that can modulate the activity of a PBH domain protein either by increasing or decreasing the activity of the calciferol and cholecalciferol. Specific examples include, without limitation, lathosterol, ergosterol, and 7-dehydro cholesterol.

The test compounds of the present invention can be obtained using any of the numerous approaches in combinatorial library methods known in the art, including: biological libraries; peptide libraries, peptoid libraries (libraries of molecules having the functionalities of peptides, but with a novel, non-peptide backbone that are resistant to enzymatic degradation but that nevertheless remain bioactive; see, e.g., Zuckermann et al. [(1994) J. Med. Chem. 37:2678-85]; spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds [Lam (1997) Anticancer Drug Des. 12:145].

Examples of methods for the synthesis of molecular libraries can be found in the art, for example in: DeWitt et al. (1993, Proc. Natl. Acad. Sci. U.S.A. 90:6909), Erb et al. (1994, Proc. Natl. Acad. Sci. USA 91:11422), Zuckermann et al. (1994, J. Med. Chem. 37:2678) Cho et al. (1993, Science 261:1303), Carrell et al. (1994, Angew. Chem. Int. Ed. Engl. 33:2059), Carell et al. (1994, Angew. Chem. Int. Ed. Engl. 33:2061), and in Gallop et al. (1994, J. Med. Chem. 7:1233).

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria (Ladner, U.S. Pat. No. 5,223,409), spores (Ladner U.S. Pat. No. 5,223,409), plasmids (Cull et al. (1992, Proc. Natl. Acad. Sci. USA 89:1865-1869) or on phage (Scott and Smith (1990, Science 249:386-390; Devlin, 1990, Science 249:404-406; Cwirla et al., 1990, Proc. Natl. Acad. Sci. 87:6378-6382; Felici, 1991, J. Mol. Biol. 222:301-310; Ladner supra).

Assays

In addition to the assays described below, assays suitable for identification of a compound that can interact with (e.g., bind and/or modulate activity of) a PHB domain polypeptide are provided in the Examples.

In one embodiment, an assay for identifying a compound that can modulate activity of a PHB domain polypeptide is a cell-based assay in which a cell that expresses a PHB domain polypeptide is contacted with a test compound, and the ability of the test compound to modulate PHB domain polypeptide activity is determined. Determining the ability of the test compound to modulate PHB domain polypeptide activity can be accomplished by monitoring, for example, binding, ion channel activity, cell lysis (e.g., in the case of RBCs), alterations in localization of the peptide or a protein that associates with the peptide. The cell, for example, can be any type of cell that can express a PHB domain polypeptide, e.g., a plant cell, a prokaryotic cell (e.g., a bacterium), a cell derived from an invertebrate such as a fly or worm, or a cell of mammalian origin, e.g., human, murine, rat, sheep, goat, pig, or non-human primate cell.

The ability of the test compound to modulate PHB domain protein binding to a compound, e.g., a cholesterol, or to bind to sterol, steroid, or cholesterol analog, can also be evaluated. This can be accomplished, for example, by labeling the compound, with a radioisotope, photoaffinity labeling, spin labeling or other suitable method such that binding of the compound, to a PHB domain polypeptide can be determined by detecting the labeled compound in a complex. Alternatively, a PHB domain polypeptide can be coupled with a radioisotope or enzymatic label to monitor the ability of a test compound to modulate PHB domain polypeptide binding to a PHB domain polypeptide ligand such as a sterol (e.g., cholesterol), steroid, or other compound having similar properties in a complex. In some cases the ligand is one that can bind to a selection of different PHB domain polypeptides derived from different PHB domain proteins. In other cases, the ligand specifically binds to the PHB domain polypeptide derived from a specific PHB domain protein. By “specifically binds” is meant a molecule that binds to a particular entity, e.g., a PHB domain polypeptide in a sample, but does not substantially recognize or bind to other molecules in the sample, e.g., a biological sample, which includes the particular entity, e.g., a PHB domain polypeptide. As used herein, a “ligand” is a compound that can bind to a polypeptide, e.g., a compound that can bind to a PHB domain polypeptide.

Methods known in the art can be used to generate a detectable label. For example, test compounds can be labeled with ¹²⁵I, ³⁵S, ¹⁴C, or ³H, either directly or indirectly, and the radioisotope detected by direct counting of radioemission or by scintillation counting. Compounds can be labeled using other methods known as the art, e.g., photoaffinity labeling.

The ability of a compound to interact with a PHB domain polypeptide with or without the labeling of any of the interactants can be evaluated. For example, the interaction of a compound with a PHB domain polypeptide can be detected, e.g., using a microphysiometer, without the labeling of either the compound or the PHB domain polypeptide (McConnell et al., 1992, Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and PHB domain polypeptide.

In general, cell-free assays involve preparing a reaction mixture of the PHB domain polypeptide and the test compound under conditions and for a time sufficient to allow the two components to interact and bind, thus forming a complex that can be removed and/or detected.

In yet another embodiment, a cell-free assay is provided in which a PHB domain polypeptide is contacted with a test compound and the ability of the test compound to bind to the PHB domain polypeptide is determined.

Because PHB domain proteins are associated with membranes, in some assays it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, isotridecypoly(ethylene glycol ether)ₙ, 3-[(3-cholamidopropyl)dimethylammonio]-1-propane sulfonate (CHAPS), 3-[(3-cholamidopropyl)dimethylammonio]-2-hydroxypropane-1-sulfonate (CHAPS), or N-dodecyl=N,N-dimethyl-3-ammonio-1-propane sulfonate.

The interaction between two molecules can also be detected, e.g., using fluorescence energy transfer (FET) (see, for example, Lakowicz et al., U.S. Pat. No. 5,631,169; Stavrianopoulos et al., U.S. Pat. No. 4,868,103; and fretimaging.org/mcnamaraintro.html). A fluorophore label on the first, ‘donor’ molecule is selected such that the donor's emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of a PHB domain polypeptide to bind to a target molecule can be accomplished using real-time Biomolecular Interaction Analysis (BIA) (e.g., Sjolander and Urbaniczky (1991) Anal. Chem. 63:2338-2345 and Szabo et al. (1995) Curr. Opin. Struct. Biol. 5:699-705]. “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the PHB domain polypeptide or the test compound is anchored onto a solid phase. The PHB domain polypeptide/test compound complexes anchored on the solid phase can be detected at the end of the reaction. In general, the PHB domain polypeptide can be anchored onto a solid surface, and the test compound (which is not anchored) can be labeled, either directly or indirectly, with detectable labels discussed herein and as are known in the art.

It may be desirable to immobilize either a PHB domain polypeptide, an anti-PHB domain polypeptide antibody, or the target molecule (ligand) of the PHB domain polypeptide, e.g., cholesterol, to facilitate separation of complexed from uncomplexed forms of one or both of the proteins, as well as to accommodate automation of the assay. Binding of a test compound to a PHB domain polypeptide, or interaction of a PHB domain polypeptide protein with a target molecule in the presence and absence of a test compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, glutathione-S-transferase/PHB domain polypeptide fusion proteins or glutathione-S-transferase/target fusion proteins can be adsorbed onto glutathione Sepharose® beads (Sigma Chemical, St. Louis, Mo.) or glutathione derivatized microtiter plates, which are then combined with the test compound or the test compound and either the non-adsorbed target protein or PHB domain polypeptide protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of PHB domain polypeptide binding or activity determined using standard techniques.

Other techniques for immobilizing a PHB domain polypeptide on matrices include using conjugation of biotin and streptavidin. Biotinylated PHB domain polypeptide can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

To conduct the assay, the non-immobilized component, e.g., a test molecule, is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways known in the art. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody). In certain assay methods, cholesterol and a test compound are both added to the immobilized PHB domain protein and the amount of bound cholesterol detected and compared to the amount of cholesterol bound in the absence of the test compound. Such assays can be used to identify compounds that can compete with cholesterol for binding to the PHB domain protein, and further, can be used to identify the affinity of the test compound for the PHB domain protein compared to cholesterol. Other forms of such competitive assays are known in the art and practitioners will understand how to apply such assays to identify test compounds and their binding affinities for a PHB domain protein.

In one embodiment, the assay is performed utilizing antibodies reactive with PHB domain protein or ligand (e.g., cholesterol) but which do not interfere with binding of the PHB domain protein to its target molecule. Such antibodies can be derivatized to the wells of the plate, and unbound ligand (e.g., cholesterol) or PHB domain polypeptide trapped in the wells by antibody conjugation. Methods for detecting such complexes, in addition to those described above for the GST-immobilized complexes, include immunodetection of complexes using antibodies reactive with the PHB domain polypeptide or ligand.

Alternatively, cell free assays can be conducted in a liquid phase. In such an assay, the reaction products are separated from unreacted components by any of a number of known techniques, including but not limited to: differential centrifugation (see, for example, Rivas et al. (1993) Trends Biochem. Sci. 18:284-7); chromatography (gel filtration chromatography, ion-exchange chromatography); electrophoresis (see, e.g., Ausubel et al., eds. Current Protocols in Molecular Biology, 1999, J. Wiley: New York.); and immunoprecipitation (see, for example, Ausubel et al., eds., 1999, Current Protocols in Molecular Biology, J. Wiley: New York). Such resins and chromatographic techniques are known to those skilled in the art (see, e.g., Heegaard (1998) J. Mol. Recognit. 11:141-8; Hage (1997) J. Chromzatogr. B. Biomed. Sci. Appl. 699:499-525). Further, fluorescence energy transfer may also be conveniently utilized, as described herein, to detect binding without further purification of the complex from solution.

In another embodiment, the assay includes contacting the PHB domain polypeptide with a known compound that binds the PHB domain polypeptide to form an assay mixture, contacting the assay mixture with a test compound, and determining the ability of the test compound to interact with PHB domain polypeptide, wherein determining the ability of the test compound to interact with PHB domain polypeptide includes determining the ability of the test compound to preferentially bind to PHB domain polypeptide, or to modulate the activity of the PHB domain polypeptide as compared to the known compound.

The PHB domain polypeptides of the invention can, in vivo, interact with one or more cellular or extracellular macromolecules, such as proteins, including interacting with such molecules to create oligomers of a PHB domain polypeptide. For the purposes of this discussion, such cellular and extracellular macromolecules are referred to herein as “binding partners.” Examples of such binding partners are proteins and other components of lipid rafts that include a PHB domain polypeptide of interest, or PHB domain protein (e.g., to form an oligomeric form of the PHB domain protein). Compounds that disrupt such interactions can be useful in regulating the activity of the PHB domain protein. Such compounds can include, but are not limited to molecules such as antibodies, peptides, and small molecules, e.g. sterols such as cholesterol, steroids, or a related molecule. The target for use in this embodiment is a PHB domain protein such as MEC-2 or Podocin. In an alternative embodiment, the invention provides methods for determining the ability of the test compound to modulate the activity of a PHB domain protein through modulation of the activity of a downstream effector of a PHB domain protein. For example, the activity of the effector molecule on an appropriate target can be determined, or the binding of the effector to an appropriate target can be determined, as previously described.

To identify compounds that interfere with the interaction between a PHB domain polypeptide and its cellular or extracellular binding partner(s), such as a non-protein organic molecule binding partner (e.g., cholesterol, a sterol, or a steroid), or a protein binding partner (e.g., Podocin and TRPC6; stomatin and TRPC1) and a reaction mixture containing the PHB domain polypeptide and a PHB domain protein binding partner is prepared (e.g., a ligand or a protein that is associated with a PHB domain protein), under conditions and for a time sufficient, to allow the two products to form complex. To test the ability of a test compound to act as an inhibitory agent, the reaction mixture is provided in the presence and absence of the test compound. The test compound can be initially included in the reaction mixture, or can be added at a time subsequent to the addition of the PHB domain polypeptide and its binding partner. Control (reference) reaction mixtures are incubated without the test compound or with a control (i.e., a known inactive compound or a known active compound). The formation of any complexes between the PHB domain polypeptide and the binding partner is then detected. The formation of a complex in the control reaction, but not in the reaction mixture containing the test compound, indicates that the compound interferes with the interaction of the PHB domain polypeptide and the interactive binding partner. Additionally, complex formation within reaction mixtures containing the test compound and normal PHB domain polypeptide can also be compared to complex formation within reaction mixtures containing the test compound and mutant PHB domain polypeptide. This comparison can be important, for example, in those cases wherein it is desirable to identify compounds that disrupt interactions of mutant but not normal PHB domain polypeptide.

These assays can be conducted in a heterogeneous or homogeneous format. Heterogeneous assays involve anchoring either the PHB domain polypeptide or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the PHB domain polypeptide and the binding partner, e.g., by competition, can be identified by conducting the reaction in the presence of the test compound. Alternatively, test compounds that disrupt preformed complexes, e.g., compounds with higher binding constants that displace one of the components from the complex, can be tested by adding the test compound to the reaction mixture after complexes have been formed. Examples of certain various formats are briefly described below.

In a heterogeneous assay system, either the PHB domain polypeptide or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

To conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the PHB domain polypeptide and the interactive binding partner is prepared in that either the PHB domain polypeptide or its binding partner is labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt PHB domain polypeptide-binding partner interaction can be identified.

In another aspect, the invention pertains to a combination of two or more of the assays described herein. For example, a modulating compound can be identified using a cell-based or a cell free assay, and the ability of the agent to modulate the activity of a PHB domain polypeptide can be confirmed in vivo, e.g., in an animal such as a C. elegans (e.g., in a touch sensitivity assay, as described in the Examples), in a X. laevis oocyte assay for ion channel effects (as described in the Examples for Podocin), or in HeLa cells (as described in the Examples for Podocin), or in animal models having a disorder associated with defective PHB domain protein activity, for example, transgenic mice having a defect in Podocin.

This invention further pertains to novel agents identified by the above-described screening assays. Accordingly, it is within the scope of this invention to further use an agent identified as described herein (e.g., a PHB domain protein modulating agent) in an appropriate animal model to determine the efficacy, toxicity, side effects, or mechanism of action, of treatment with such an agent. Furthermore, novel agents identified by the above-described screening assays can be used for designing treatments for disorders associated with defective PHB domain protein activity.

The invention also relates to methods of identifying subjects having a disorder related to a PHB domain protein (e.g., Podocin or an MEC-2 protein) and predicting whether such subjects will respond to treatment with a lipid-interfering compound, e.g., a compound that modulates the interaction of such a molecule with a PHB domain protein (discussed infra). Thus, mutation screening in cholesterol binding portions of PHB domain proteins can be used as part an individualized approach to treating patients with PHB domain protein-related diseases.

### Compositions

The invention also relates to compositions comprising an isolated PHB domain sequence that can bind to PBH domain ligand, e.g., cholesterol. In some cases, the isolated domain sequence includes up to 5 additional amino acids (termed herein “extended PHB domains”). Non-limiting examples of specific PHB domains include amino acids 125-286 of murine Podocin and amino acids 129-300 from C. elegans MEC-2. Non-limiting examples of extended PHB domains include amino acids 120-186 of murine Podocin and amino acids 124-300 of C. elegans MEC-2. Such compositions are useful in assays to identify compounds that can bind to the PHB domain sequence.

### Diagnostic, Prognostic, and Theranostic Assays

The presence, level, or absence of a PHB domain protein in a biological sample can be evaluated by obtaining a biological sample from a test subject and contacting the biological sample with a compound or an agent capable of detecting the activity of the PHB domain protein in the biological sample. The term “biological sample” includes tissues, cells and biological fluids isolated from a subject, as well as tissues, cells and fluids present within a subject. A biological sample can be, without limitation, a tissue biopsy specimen or blood. A biological sample can also be from a prokaryote such as a bacterium or from a plant.

In another embodiment, the methods include contacting a control sample (a reference) with a compound or agent (e.g., a PHB domain polypeptide ligand such as a sterol, cholesterol, or related compound) that can bind to the PHB domain polypeptide, and comparing the binding in the control sample with the binding in the test sample. A difference between the reference and the test sample can indicate a disorder associated with defective activity of the PHB domain polypeptide.

The invention also includes kits for detecting the presence of PHB domain polypeptide activity in a biological sample. For example, the kit can include a compound that can bind to a PHB domain polypeptide (e.g., the natural ligand for a PHB domain protein such as cholesterol) and a standard (reference). The compound or agent can be packaged in a suitable container. The kit can further comprise instructions for using the kit to detect PHB domain protein binding or activity.

For antibody-based kits, the kit can include: (1) a first antibody (e.g., attached to a solid support) which binds to a PHB domain polypeptide; optionally, (2) a ligand that can bind to the PHB domain protein, and optionally, (3) a second, different antibody that can detect ligand bound to the PHB domain polypeptide.

The diagnostic methods described herein can be used identify subjects having, or at risk of developing, a disease or disorder associated with aberrant or unwanted PHB domain protein activity. As used herein, the term “unwanted” includes an unwanted phenomenon involved in a biological response such as cell lysis or defective regulation of one or more ion channels.

In one embodiment, a disease or disorder associated with aberrant or unwanted PHB domain protein activity is identified. A test sample is obtained from a subject and PHB domain protein is evaluated, wherein aberrant or otherwise undesirable PHB domain protein activity is diagnostic for a subject having or at risk of developing a disease or disorder associated with aberrant or unwanted PHB domain protein activity. As used herein, a “test sample” refers to a biological sample obtained from a subject of interest, including a biological fluid (e.g., blood), cell sample, or tissue sample.

The prognostic assays described herein can be used to determine whether a subject can be administered an agent (e.g., a compound that can act as a ligand that increases PHB domain protein activity or a compound that can act as a ligand that decreases PHB domain protein activity) to treat a disease or disorder associated with aberrant or unwanted PHB domain protein activity. For example, such methods can be used to determine whether a subject can be effectively treated with an agent for a disorder involving cell lysis or aberrant ion channel activity.

Other useful assays include diagnostic assays that identify whether a mutation is in a PHB domain of a protein. Identification of such a mutation can be useful for determining treatment of a condition associated with the PHB domain protein. For example, if a mutation associated with an undesirable condition is identified in a PHB domain of a subject, treatment with a compound that is directed to the PHB domain can be useful or otherwise ameliorates the undesirable effect of the mutant PHB domain can be administered to the subject. In addition, if the defect is identified in the PHB domain of a specific protein, a treatment can be selected that comprises a compound that specifically targets the PHB domain of the specific protein (i.e., does not target all PHB domains in a cell).

### Compositions

The invention also relates to compositions comprising a PHB domain sequence. In some cases, the PHB domain sequence includes the hydrophobic domain that is at the amino terminus of a naturally occurring PHB domain sequence. In certain embodiments, the PHB domain sequence includes up to 5 additional amino acids at the amino terminus, e.g., 4 additional amino acids, 3 additional amino acids, 2 additional amino acids, or 1 additional amino acid. In general, the additional amino acids include at least one, two, three, four, or five hydrophobic amino acids. In certain cases, the additional amino acids include a proline. In other cases, there is no proline in the additional amino acid sequence.

### EXAMPLES

The invention is further illustrated by the following examples, which should not be construed as limiting.

### Example 1

### Materials and Methods

Coimmunoprecipitation. Immunoprecipitations were performed as described in the art. Briefly, HEK 293T cells were transiently transfected by the calcium phosphate method. After incubation for 24 hours, cells were washed twice and lysed in a 1% Triton X-100 lysis buffer. After centrifugation (15,000×g, 15 minutes, 4° C.) and ultracentrifugation (100,000×g, 30 minutes, 4° C.) cell lysates containing equal amounts of total protein were incubated for one hour at 4° C. with the appropriate antibody, followed by incubation with 40 μl of protein G-Sepharose® beads for approximately three hours. The beads were washed extensively with lysis buffer, and bound proteins were resolved by 10% SDS-PAGE.

Preparation of Detergent-Resistant Membrane Domains and Cholesterol Measurement—For preparation of low-density Triton® X-100—insoluble membrane domains (DRM; LDF) HEK 293T cells were homogenized by 20 strokes in a Dounce homogenizer in 1 ml of MBS buffer (250 mM NaCl, 5 mM EDTA, 10 mM Tris, pH 7.4, proteinase inhibitors) in the presence of 1% Triton® X-100 and centrifuged for ten minutes at 3,000×g at 4° C. The lysates were incubated for 45 minutes on ice in the presence of 1% Triton® X-100, adjusted to 45% sucrose and pipetted at the bottom of an ultracentrifuge tube. Samples were then overlaid with a sucrose step gradient (2 ml of 30% sucrose and 1 ml of 5% sucrose in MBS) as described previously {Huber, 2003 #205}. Gradients were centrifuged for 20 hours at 200,000×g at 4° C. in a swing-out rotor, and seven fractions (700 mL each) were collected starting from the top and analyzed by SDS-PAGE. Cholesterol was measured using the Amplex® Red Cholesterol Assay Kit (Molecular Probes).

Digitoninin Precipitation Assay—The assay was performed essentially as described in the art (Charrin et al. (2003) Eur. J. Immunol. 2003.33:2479-2489). For precipitation of cholesterol with digitonin, a 1/10th volume of 10% digitonin in methanol, or methanol as a control, was added to the Brij® 97 or Triton® X-100 supernatants. After 30 minutes at 4° C., the insoluble material was separated by centrifugation, washed once with lysis buffer supplemented with methanol or digitonin and resuspended four times concentrated in loading buffer for further analysis on immunoblots.

Photoaffinity labeling. Experiments were performed as described infra. For labeling with [3H]photocholesterol or [3H]photophosphatidylcholine, cells were supplemented with delipidized FCS and [3H]photocholesterol-MBCD complex or 10-azi-stearate and [3H]choline were added. After 16 hours, cells were washed three times with Ca/Mg-PBS (PBS containing 0.1 mM CaCl₂ and 1 mM MgCl₂) and irradiated for 30 minutes in Ca/Mg-PBS with a 100 W mercury lamp. For further analysis, cells were lysed on ice for 1 h in lysis buffer (50 mM Tris-HCl pH 7.4, 140 mM NaCl, 5 mM EDTA, 0.5 mM phenylmethylsulfonyl fluoride (PMSF), 1% Triton® X-100, 0.5% deoxycholate). The lysate was centrifuged for eight minutes at 10,000×g, and the supernatant was subjected to immunoprecipitation using an anti-FLAG monoclonal antibody. Aliquots of the starting extract, the supernatants after immunoprecipitation and the immunoprecipitates were separated by SDS-PAGE (12%) followed by blotting of the proteins on PVDF membrane. Radioactivity was detected by phosphoimaging (Fujifilm BAS-2500, Fuji Photo Film Co., Ltd, Japan) using a Tritium-sensitive BAS-TR2025 imaging plate (Fuji Photo Film Co., Ltd, Japan). From the same membrane, an immunoblot was performed using anti-FLAG antibodies.

Reagents and Plasmids. Mouse Podocin cDNA constructs have been described previously [Huber et al. (2003) Hum. Mol. Genet. 12:3397-405; Huber et al. (2001) J. Biol. Chem. 18:18]. TRPC6 was cloned from a human podocyte cDNA library. MEC-2 cDNA was cloned from a C. elegans ORF AAA87552 (Open Biosystems). Truncations and mutations of Podocin, MEC-2, and TRPC6 were generated by standard cloning procedures. All other constructs have been described previously [Goodman et al. (2002) Nature 415:1039-42; Huber et al. (2001) J. Biol. Chem. 18:18]. Some experiments involving MEC-2 had to be performed with αENaC, a mammalian ENaC protein, instead of MEC-4 because MEC-4 cDNA did not express well in HEK 293T cells. Antibodies have been described or were obtained from Sigma (St. Louis, Mo.; anti-FLAG M2), Alomone (Jerusalem, Israel) and Chemicon (Millipore, Billerica, Mass.) (anti-TRPC6), and Serotec (Kingston, N.H.; anti-V5). Bacterial vectors for the expression of His-tagged recombinant proteins fused to the C-terminus of NusA were obtained from G. Stier (EMBL Heidelberg).

Cell Culture Studies. Most cell studies used HEK 293T cells that were grown in DMEM as described [Huber et al. (2001) J. Biol. Chem. 18:18]. Cholesterol-depleted cells were prepared by growing cells in DMEM with pravastatin (8 μM) for two days and then methyl-β-cyclodextrine (MBCD; 5 mM) for 30 minutes just before the experiment. Immunoprecipitations from HEK 293T cells were performed as described [Huber et al. (2001) J. Biol. Chem. 18:18]. Palmitate labeling, the digitonin precipitation assay [Charrin et al. (2003) Eur. J. Immuno. 33:2479-89] and photoaffinity labeling [Thiele et al. (2000) Nat. Cell Biol. 2:42-9] were performed as described. Expression and purification of recombinant proteins was as described in Benzing et al. [(1999) Nat. Med. 5:913-8].

PHB protein multimerization was studied by velocity gradient centrifugation and blue native gel electrophoresis [Schagger et al. (1991) Anal. Biochem. 199: 223-31]. For preparation of Podocin multimeric complexes HEK293T cells were lysed in 1 ml of Mes-buffered saline (MBS) in the presence of 1% Triton® X-100 and centrifuged for 10 minutes at 1,000×g at 4° C. After centrifugation, the supernatant was collected and SDS was added at a final concentration of 0.1% and incubated for 20 minutes on ice. Thereafter, the lysate was cleared by centrifugation for 15 minutes at 100,000×g. Four milliliters of a discontinuous sucrose gradient (40-5%) was layered on top of a 60% sucrose cushion in a Beckman ultracentrifuge tube. One milliliter of the cell lysate was adjusted with 1 ml of MBS, added on top of this gradient and subjected for centrifugation for 16 hours at 180,000×g at 4° C. in a Beckman SW-41 rotor. After centrifugation 2 ml of the supernatant were discarded and 8 fractions (500 μl each) were collected starting from the top and analyzed by SDS-PAGE.

In vitro cholesterol interaction. Podocin truncations were cloned into various bacterial expression vectors and tested for the expression of soluble recombinant fusion proteins. Expression as His-tagged proteins fused to the C-terminus of NusA (vectors obtained from Gunter Stier, EMBL, Heidelberg) resulted in a large fraction of soluble recombinant Podocin protein that could be affinity purified on Ni+ columns. Purity of the preparation was confirmed on Coomassie gels. For in vitro cholesterol interaction assays 2-20 μg of affinity purified Podocin protein was bound to 30 μl of Ni+ beads and incubated with 0.1 μCi [³H]cholesterol (Amersham) complexed with low amounts of cyclodextrine. After binding for 10 minutes at 37° C. beads were washed extensively and counted in a scintillation counter. To confirm equal loading of the beads aliquots of the bound protein were run on Coomassie gels. Competition experiments were performed with 1 μg of affinity purified Podocin protein fused to NusA or NusA alone as a control. Samples were incubated with radioactively labeled cholesterol in the absence (no cold competition) or presence (cold competition) of varying amounts of [³H]cholesterol. Cold competition was approximately 100-fold excess of cold cholesterol. The sample was washed extensively and subjected to scintillation counting.

C. elegans experiments. C. elegans strains were cultured at 20° C., assayed for touch sensitivity, and prepared for immunofluorescence as described previously [Zhang et al. (2004) Curr. Biol. 14:1888-96; Goodman et al. (2002) Nature 415:1039-42]. Briefly, C. elegans strains were cultured at 20° C. as described in the art. Strains were grown for at least three consecutive generations without starvation before carrying out the experiments described herein. Gentle touch and harsh touch sensitivity were assayed in blind tests by stimulating with an eyebrow hair or prodding with a platinum wire, respectively, as described in the art. To quantify the response, the number of responses in about 30 animals to 10 touches delivered alternately near the head and tail was recorded. Whole-mount immunofluorescence microscopy was carried out using methods known in the art.

Media for growth on limiting or no cholesterol or on other sterols were prepared from chloroform-extracted reagents as described by Matyash et al. [(2004) PLoS Biol. 2: e280].

Oocyte Electrophysiology. Xenopus laevis oocytes were isolated from adult frogs (Kähler, Hamburg, Germany), dispersed and defolliculated by a 45 minute treatment with collagenase (type A, Boehringer, Germany), and were rinsed and kept at 18° C. in ND96-buffer (in mmol/l): NaCl 96, KCI 2, CaCl₂ 1.8, MgCl₂ 1, HEPES 5, Na-pyruvate 2.5, pH 7.55), supplemented with theophylline (0.5 mmol/l) and gentamycin (5 mg/l).

To prepare cRNAs for TRPC6, Podocin and Podocin-DPHB (Podocin-ΔPHB), cDNAs encoding TRPC6, Podocin and Podocin-DPHB were transcribed in vitro using the T7 promoter and polymerase (Promega, Madison, Wis.). After isolation from adult Xenopus laevis female frogs (Kähler, Hamburg, Germany), oocytes were dispersed and defolliculated by a 45 minute treatment with collagenase (type A, Boebringer, Germany). Subsequently, the oocytes were rinsed and kept at 18° C. in ND96-buffer (in mmol/l): NaCl 96, KCI 2, CaCI2 1.8, MgCI2 1, HEPES 5, Na-pyruvate 2.5, pH. 7.55), supplemented with theophylline (0.5 mmol/l) and gentamycin (5 mg/1). cRNAs (1-10 ng) for TRPC6, Podocin and Podocin-ΔPHB were transcribed in vitro from cDNAs using the T7 promoter and polymerase (Promega, USA) and injected into oocytes after dissolving in 47 nl double-distilled water (Nanoliter Injector WPI, Germany). Water injected oocytes served as controls. Two to four days after injection, oocytes were impaled with two electrodes (Clark instruments) that had resistances of <1 MΩ when filled with 2.7 mol/l KCI. Using two bath electrodes and a virtual-ground headstage, the voltage drop across Rₛₑᵣᵢₐₗ was effectively zero. Membrane currents were measured by voltage clamping of the oocytes (Warner oocyte clamp amplifier OC725C) in intervals from −60 to +40 mV, each 1 s. Conductances were calculated according to Ohm's law. Na⁺ conductances were determined by replacing Na⁺ by n-methyl-D-glucamine (G_{NMDG}) in a Ca²⁺ free bath solution, before and after stimulation with 10 μM dioctanoyl glycerol (Sigma, Germany). During the entire experiment, the bath was continuously perfused at a rate of 5-10 ml/minute. All experiments were conducted at room temperature (22° C.).

Ca²⁺-FLIPR Assay—HeLa-Cx43 cells were loaded with 4 mM FLUO-4/AM and 0.04% Pluronic F-127 (both from Molecular Probes) in HBS but with 20 mM HEPES and 2.5 mM probenecid as described previously (20). After loading, cells were washed twice with HBS by an automated plate washer (Denley Cellwash, Labsystems) and transferred to the FLIPR (Molecular Devices). The FLIPR integrates an argon laser excitation source, a 96-well pipettor, and a detection system utilizing a CCD imaging camera. Fifty milliliters of 3× histamine concentrations were delivered within 2 seconds simultaneously to all of the wells containing 100 ml of HBS. Fluorescence emissions from the 96-wells were monitored simultaneously at an emission wavelength of 515 nm after excitation with 488 nm (F488). Fluorescence data were collected at 0.25 Hertz, 48 seconds before and 8 minutes after stimulation, and analyzed off-line. Histamine response amplitudes were determined from the fluorescence at 3.2 minutes (peak of calcium influx) of the solvent control-subtracted and base-line-corrected traces, and averaged over 3-4 wells of same transfectants, receiving the same stimulus. EC50 values and curves were derived from fitting the function f(x)=(a−d)/(1+(x/C)nH)+d to the data by non-linear regression with a=minimum, d=maximum, C=EC50, and nH=Hill coefficient.

Statistical Analysis. Data were expressed as mean±SEM of n experiments. Statistical evaluation was performed using Student's t test or ANOVA for repeated measures, followed by a Bonferroni test as post-test (SigmaPlot, Jandel Scientific and Instat2, GraphPad). Values of p<0.05 were considered to be statistically significant.

### Example 2

### Multimeric Complexes

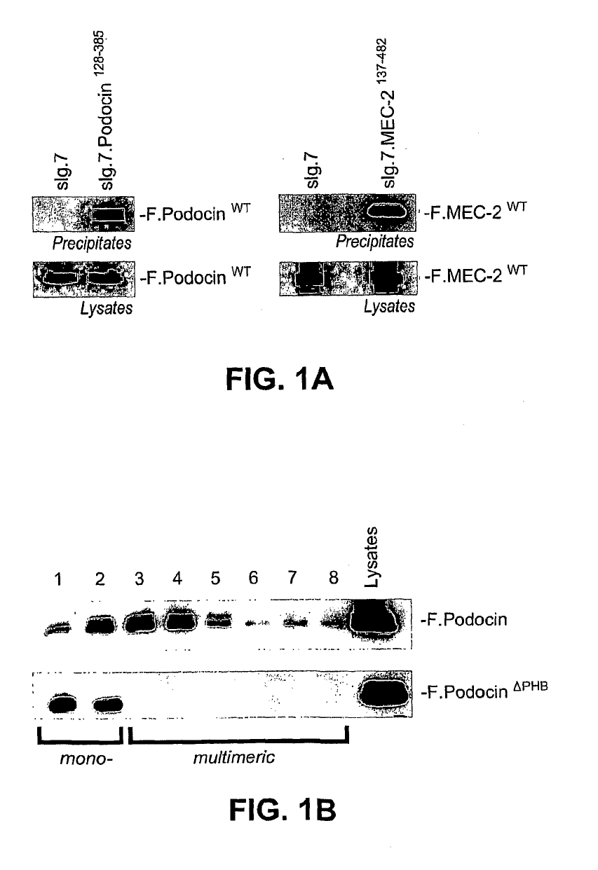

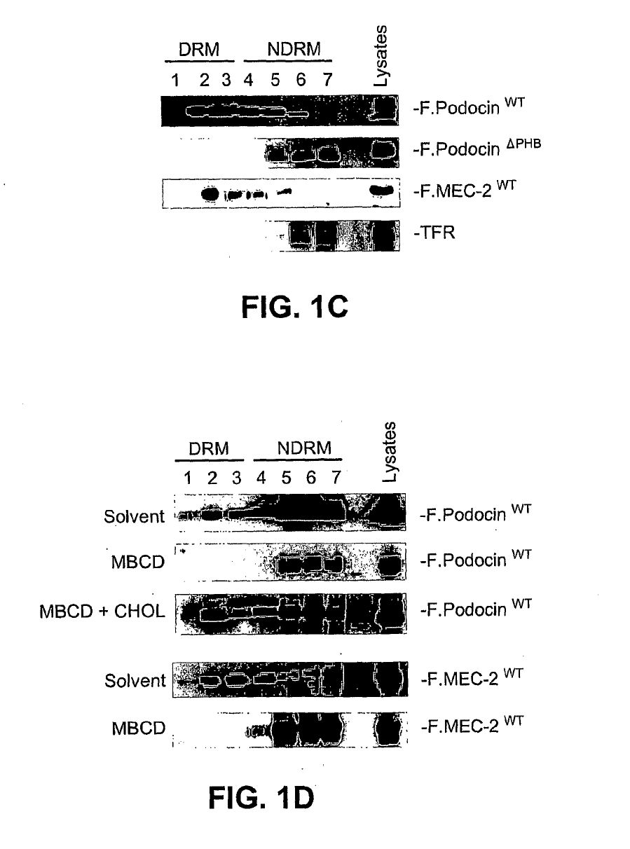

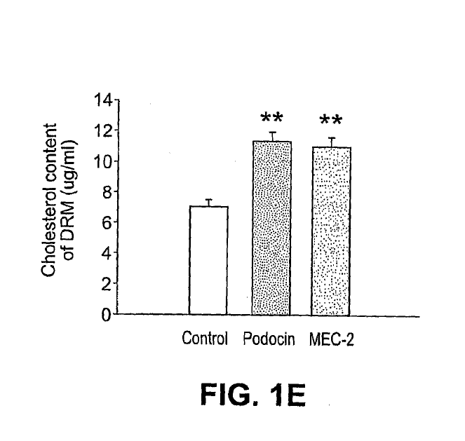

Co-immunoprecipitation was used to investigate the tertiary features of Podocin and of MEC-2. In immunoprecipitation experiments, FLAG-tagged Podocin (F.Podocin) and FLAG-tagged MEC-2 (F.MEC-2) coimmunoprecipitated with sIg.7-tagged MEC-2 and Podocin but not with a control protein (sIg.7) (FIG. 1A). These data demonstrated that that Podocin and MEC-2 homo-oligomerize. This method can be used to assay whether a test compound can modulate the oligomerization of a PHB domain polypeptide by incubating a test compound with the polypeptide and assaying by immunoprecipitation whether the test compound modulates immunoprecipitation.

This assembly of high-molecular weight complexes by homophilic interactions required the PHB domain, as demonstrated by velocity gradient centrifugation after detergent lysis. In these experiments, velocity gradient centrifugation was performed after mild detergent solubilization of the samples (FIG. 1B). This method can be used to determine whether a test compound can affect multimerization, e.g., by incubating a cell expressing a selected PHB domain protein with a test compound, and assaying multimerization using velocity density gradient centrifugation.

Equilibrium density gradient centrifugation of detergent-solubilized membrane lysates from either Podocin or MEC-2-expressing cells revealed that the complexes migrated towards low density fractions, representing detergent-resistant membrane domains with light buoyant density (FIG. 1C). In these experiments, MEC-2 and Podocin multimeric complexes displayed a low buoyant density and associated with the detergent-resistant light fractions (DRM; LDF) in density gradient centrifugation of Triton® X-100 solubilized membranes. Transferrin receptor (TFR) is a known NDRM protein, and was used to demonstrate the purity of the preparation. Other PHB domain proteins have been shown to be associated with the low density fraction.

The enrichment of Podocin in DRM was critically dependent on the PHB domain; a deletion mutant of Podocin lacking the PHB domain (PodocinDPHB) did not target to DRM but cofractionated with a non-DRM (NDRM) marker, transferrin receptor (TFR) (FIG. 1C). Confocal microscopy as well as biochemical assays revealed that this deletion mutant, which still contains the hydrophobic membrane-penetrating region, localizes to the plasma membrane. To test whether detergent resistance and light density could be attributed to cholesterol attached to these multimeric complexes, Podocin-expressing cells were treated with methyl-β-cyclodextrin (MBCD), which extracts cholesterol from the plasma membrane. Limited cholesterol depletion resulted in the loss of Podocin and MEC-2 from the DRM fractions without affecting Podocin/MEC-2 protein levels at the plasma membrane indicating that cholesterol plays a role in DRM targeting. This effect could be reversed by the readdition of cholesterol to the MBCD-treated cells (FIG. 1D). These data demonstrate that Podocin and MEC-2 form homo-oligomeric complexes with light buoyant density.

The appearance of Podocin/MEC-2 complexes in DRM could result from the targeting of the proteins to preexisting cholesterol-rich domains or the recruitment of free cholesterol to these protein complexes. To investigate these alternatives, cholesterol levels were measured in DRM fractions derived from Podocin- and MEC-2-expressing cells. Total cholesterol content was not affected by the expression of Podocin or MEC-2. In contrast, Podocin and MEC-2 expression markedly increased the amount of cholesterol in the DRM fractions (FIG. 1E). These data indicate that Podocin and MEC-2 complexes have a low buoyant density, not because they are recruited to preexisting lipid domains, but because they contribute to the de novo formation of protein-lipid complexes.

### Example 3

### Podocin and MEC-2 are Cholesterol-Binding Proteins

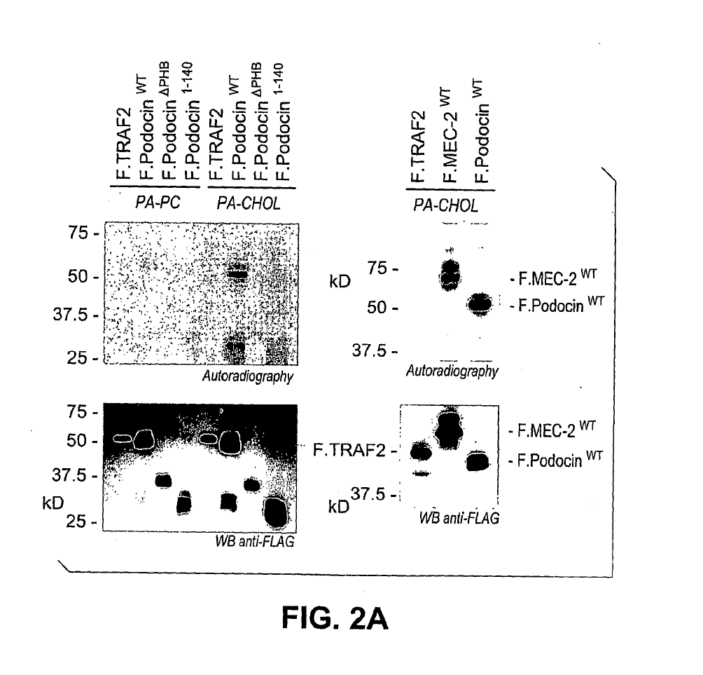

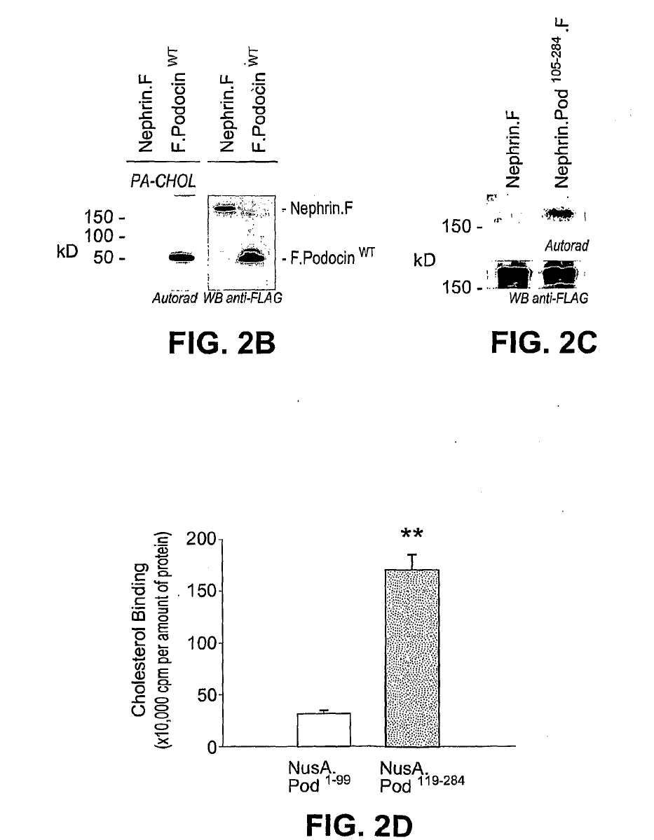

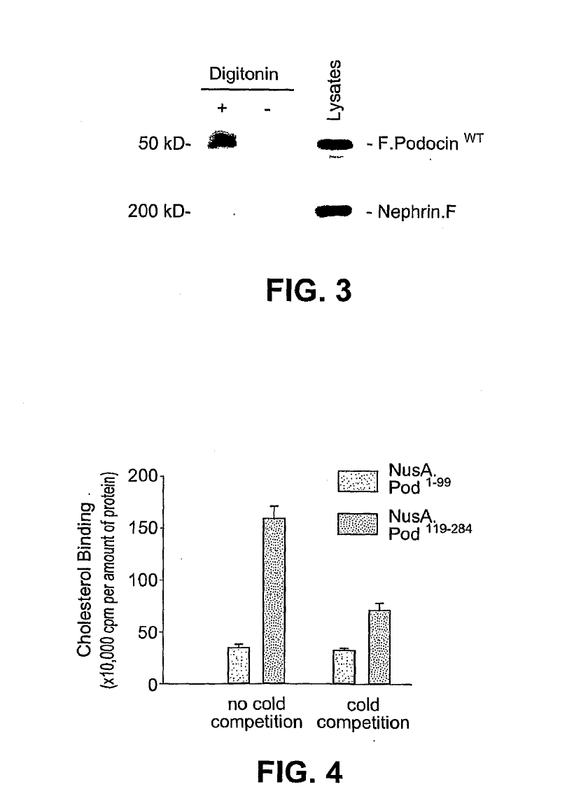

Podocin [Huber et al. (2003) Hum. Mol. Genet. 12: 3397-405] and several other PHB-domain proteins are found in cholesterol-rich membrane fractions. To test whether Podocin and MEC-2 bind to cholesterol, both proteins were expressed in HEK 293T cells and tested for binding of photoactivatable lipids (FIG. 2A). These derivatives attach to associated molecules when they are stimulated by UV light [Thiele et al. (2000) Nat. Cell Biol. 2:2-9]. Podocin and MEC-2 bound cholesterol but not phosphatidylcholine (FIG. 2A). Cholesterol binding required the PHB-domain, since Podocin lacking this domain (Podocin^{ΔPHB}) did not label. Binding was quite specific since other membrane proteins such as the immunoglobulin superfamily member and Podocin-interacting protein Nephrin were not labeled (FIG. 2B). To confirm these data, digitonin precipitation experiments were conducted. The experiments were conducted essentially as described in Charrin et al. [(2003) Eur. J. Immunol. 33:2479-2489]. Because digitonin precipitation depends on sterols these data confirm that, Podocin, but not Nephrin bind to sterols. (FIG. 3).

The importance of different regions of Podocin for cholesterol binding was tested by fusing them to the extracellular and transmembrane domains of Nephrin. A fusion containing the PHB-domain bound cholesterol, but more efficient cholesterol binding was observed when the PHB-domain and the adjacent N-terminal hydrophobic domain were included (FIG. 2C). To ensure that cholesterol labeling was the result of direct binding and did not occur through passive stochastic attachment of cholesterol at the cell membrane, fragments of Podocin were produced in E. coli and their ability to bind [³H]cholesterol in vitro was tested. In these experiments, testing in vitro binding of cholesterol, NusA or NusA fused to the cholesterol binding domain of Podocin (amino acids 119-284) was incubated with radioactively labeled cholesterol, washed extensively and subjected to scintillation counting. It was found that the PHB-domain was sufficient for cholesterol binding but binding was more efficient when the polypeptide included the PHB-domain and the N-terminal adjacent hydrophobic sequence (FIG. 2D). To test specificity, binding experiments were performed in which NusA or NusA fused to the cholesterol binding domain of Podocin was incubated with radioactively labeled cholesterol in the absence (no cold competition) or presence (cold competition) of an approximately 100-fold excess of unlabeled cholesterol, washed extensively, and subjected to scintillation counting. It was found that binding was competed with approximately 100× excess of cold cholesterol (FIG. 4).

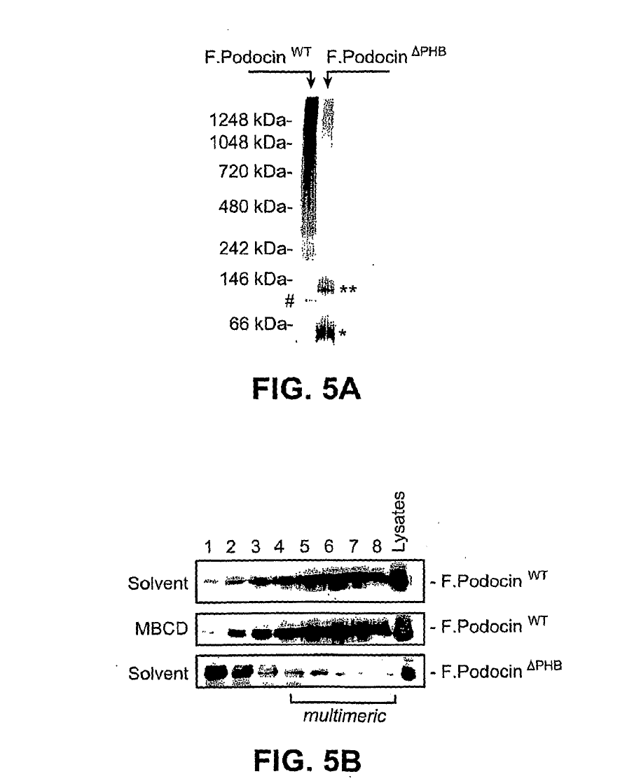

Previously it was shown that shown that Podocin homo-oligomerizes and forms high-molecular weight complexes by homophilic interactions that require the PHB-domain [Huber et al. (2003) Hum. Mol. Genet. 12:3397-405]. MEC-2 also homo-oligomerizes (infra) as do other PHB-domain proteins [Umlauf et al. (2006) J. Biol. Chem.]. To investigate the size of complexes, cellular lysates were subjected to blue native gel electrophoresis and FLAG-tagged Podocin and the mutant FLAG-tagged Podocin^{ΔPHB} were identified. The size of the wild type Podocin complexes suggested that they contain at least 20 to 50 molecules (FIG. 5A). To investigate whether multimerization requires cholesterol binding, cell lysates were subjected to velocity gradient centrifugation after mild detergent solubilization. It was found that multimerization, however, does not require cholesterol binding. Limited cholesterol depletion with methyl-β-cyclodextrin (MBCD) of Podocin-expressing cells in these experiments did not interfere with the formation of high-molecular weight complexes (FIG. 5B). Thus, Podocin and MEC-2 bind cholesterol, themselves, as well as other proteins.

### Example 4

### Touch Sensitivity Requires Sterol Binding by MEC-2 in C. elegans

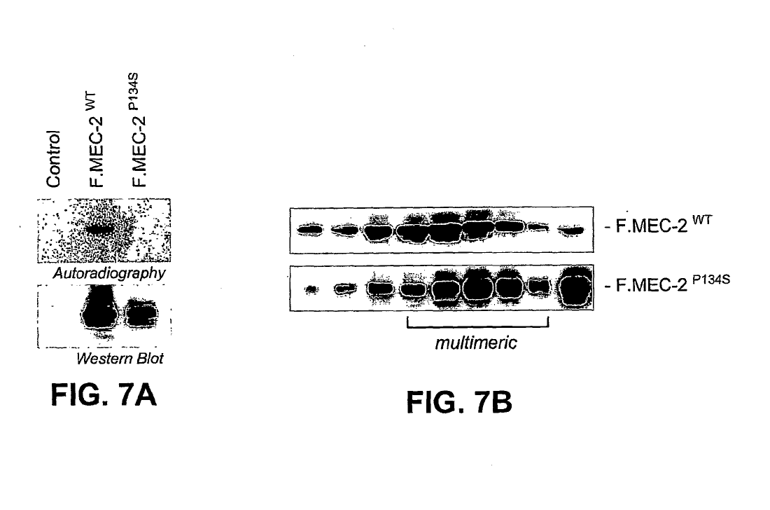

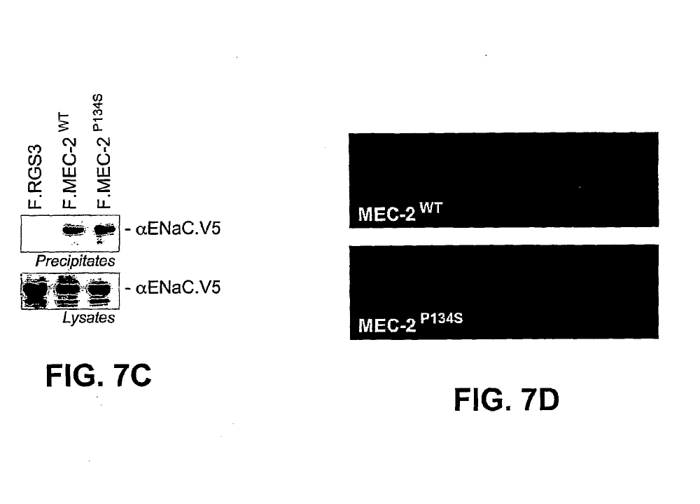

To test the in vivo importance of cholesterol binding, the requirement for MEC-2 in C. elegans touch sensitivity was exploited. Twenty-three mec-2 alleles causing touch insensitivity in C. elegans have missense mutations [Zhang et al. (2004) Curr. Biol. 14:1888-96], and most of the resulting proteins were screened for their ability to bind cholesterol, localize to the membrane, multimerize, and interact with associated channels. Cholesterol binding was absent in some mutants and reduced in many others. As an example, the protein MEC-2(P134S) was studied. This protein is produced by the u274 allele. This mutation substitutes a serine for proline in the hydrophobic region preceding the PHB-domain (FIG. 6B). Worms expressing the mutant allele are completely touch insensitive (two of 50 animals responded once to five touches). In experiments testing the ability of the mutant to bind cholesterol, photoaffinity cholesterol labeled FLAG-tagged wild-type MEC-2 but not MEC-2(P134S) (FIG. 7A), even though it localized to the plasma membrane, multimerized (FIG. 7B), and interacted with the MEC-4-related channel αENaC (FIG. 7C). These experiments demonstrate that MEC-2 can bind to other DEG/ENaC proteins and that the mutant binding does not depend on cholesterol.

Overall these data indicate that loss of touch sensitivity results from the loss of cholesterol binding of this protein. Furthermore these data are consistent with a role for MEC-2 in recruiting or maintaining cholesterol in the multiprotein MEC-4 channel complex in vivo, although previously localized cholesterol could assist in the association of MEC-2 with the complex. These data also illustrate an assay method that can be used for identifying compounds that modulate the activity of a PHB domain protein. For example a compound that is a PHB domain modulator in an in vitro assay can be tested for its effect in C. elegans. Administration of such a compound to a wild type animal may result in an animal with a phenotype similar to that of an animal mutant in a MEC-2 gene. Compounds can also be identified that can rescue the mutant phenotype. Such compounds are useful as candidate compounds for treating conditions related to aberrant activity of a PHB domain protein (e.g., MEC-2 or an ortholog of MEC-2). In addition, a test compound can be localized as discussed supra. A compound that binds to a PHB binding domain and inhibits the effect of cholesterol bound to such a protein will, in some cases, exhibit a localization pattern similar to the localization pattern for cholesterol in the mutant.

To investigate whether the mutant protein localizes correctly in touch channel puncta of touch neurons in the nematode, wild type and mutant animals were stained with antibodies directed against MEC-2. These experiments detected localization of wild type and mutant MEC-2 in processes of touch receptor neurons in C. elegans. Both proteins were found in the process (suggesting that both localize to the plasma membrane), but the P134S protein was not found in the characteristic puncta formed by the mechanosensory channel complex. The u274 mutation did not prevent the localization of the mutant protein to the plasma membrane (FIG. 7D). However, the distribution of MEC-2(U247) within the plasma membrane was not the same as in wild-type animals since the protein was not found in puncta (0/50 animals) but was more uniformly distributed. These data are consistent with a role for cholesterol binding in the formation of higher order structures at the independently localized MEC-4 puncta in vivo.

If cholesterol, or a cholesterol derivative [Chitwood (1999) Crit. Rev. Biochem. Mol. Biol. 34:273-84], is needed for channel function, cholesterol-deprived worms should be touch insensitive. However, when wild-type C. elegans larvae were transferred from normal (13 μm) cholesterol to cholesterol-depleted plates, they produced F1 progeny that arrested as young larvae and that were touch sensitive. Presumably, these arrested larvae were not completely depleted of cholesterol, having, as shown below, sufficient cholesterol for touch sensitivity but not enough for further development.

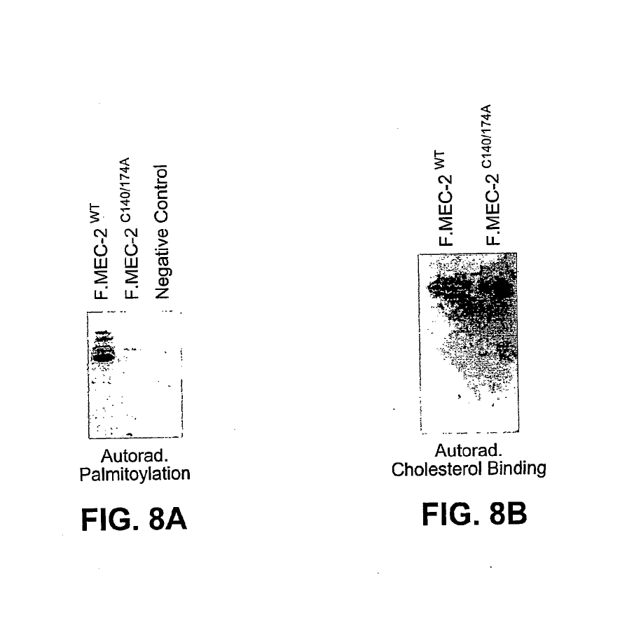

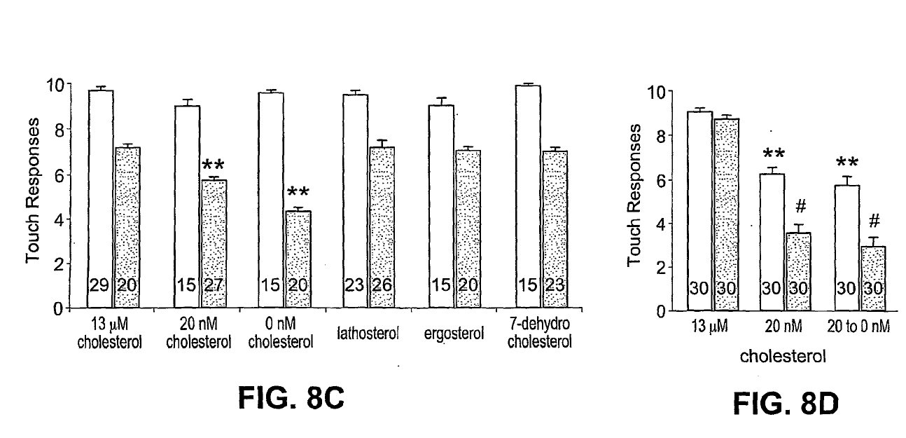

The need for cholesterol in C. elegans touch sensitivity was demonstrated in two ways. First, a version of MEC-2 with reduced cholesterol binding was generating by mutating the predicted palmitoylation sites of the protein. Briefly, HEK293T cells were transfected with wild type MEC-2 or MEC-2(C140/174A) and labeled with [³H]palmitic acid or [³H]photoaffinity cholesterol. Equal expression of proteins in the lysates was confirmed on Western blots. Substitution of alanine for cysteine at amino acids 140 and 174 resulted in the loss of palmitoylation (FIG. 8A) and a reduction of cholesterol binding (FIG. 8B). These alterations did not affect overall protein levels, multimerization, or localization to the plasma membrane, but mec-2 null worms expressing the mec-2(C140/174A) gene were conditionally dependent on cholesterol for touch sensitivity. These animals showed virtually the same touch sensitivity as wild-type animals on plates with normal amounts of cholesterol but reduced touch sensitivity when grown on cholesterol-free plates (FIG. 5C and FIG. 8D). This defect was dependent on the cholesterol concentration and could be rescued by substituting lathosterol, ergosterol, and 7-dehydro cholesterol for cholesterol in the growth medium (FIG. 8C). Because touch sensitivity of wild-type animals was not affected by this limited cholesterol depletion, the effects observed with the palmitoylation mutant cannot be attributed to indirect effects on neuronal growth or development. Test compounds that target palmitoylation sites in PHB domain proteins can be used to modulate activity of such proteins, for example, by reducing their activity. These data also demonstrate that in the case of an organism having a mutation in a PHB domain, e.g., in a palmitoylation site, screening for compounds that increase activity of the mutation can be identified.

The second demonstration of cholesterol dependence was seen when cholesterol levels were lowered further by transferring animals grown on minimal (20 nM) cholesterol plates for three generations (it was found that the animals arrest their development after about four generations) to 0 nM cholesterol plates. Larvae placed on minimal or zero cholesterol never became adults, but arrested in their development. The animals were noticeably more debilitated (many could not move) under these conditions. Nonetheless, wild-type animals that showed normal movement had become relatively insensitive to touch and animals with the MEC-2 palmitoylation mutations were even less sensitive to touch (FIG. 8D). These data demonstrate that touch sensitivity is dependent on sterols in vivo and suggest that sterols recruited to the MEC-4 channel complex by MEC-2 are needed for its function.

Methods using phenotypic responses that are the result of interference with the function of a PHB domain mutation (such as touch sensitivity) can be used to assay compounds that can restore or improve normal function or as references to demonstrate the expected effect of a compound that interferes with the activity of a PHB domain polypeptide.

### Example 5

### Podocin-Mediated Regulation of TRPC Channel Activity

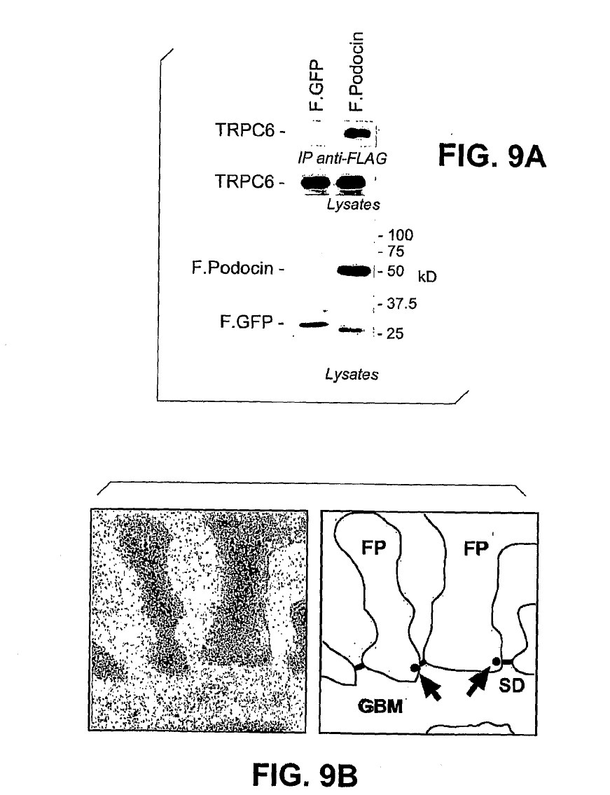

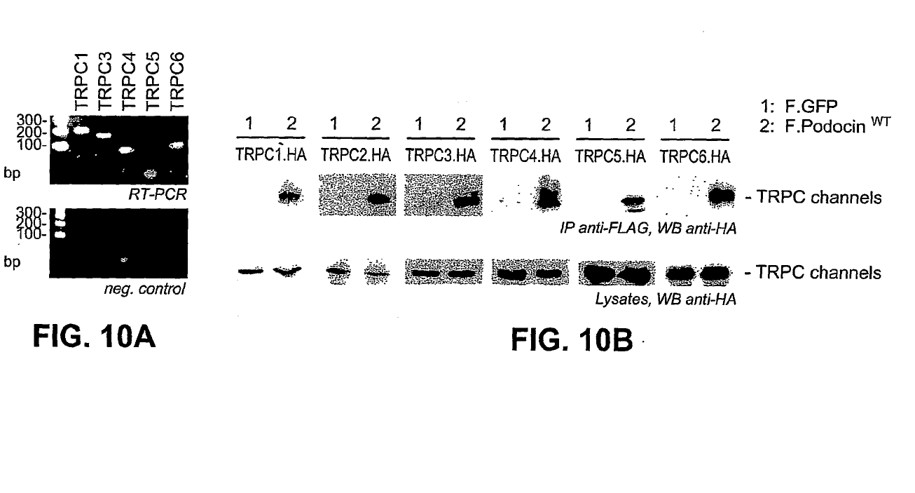

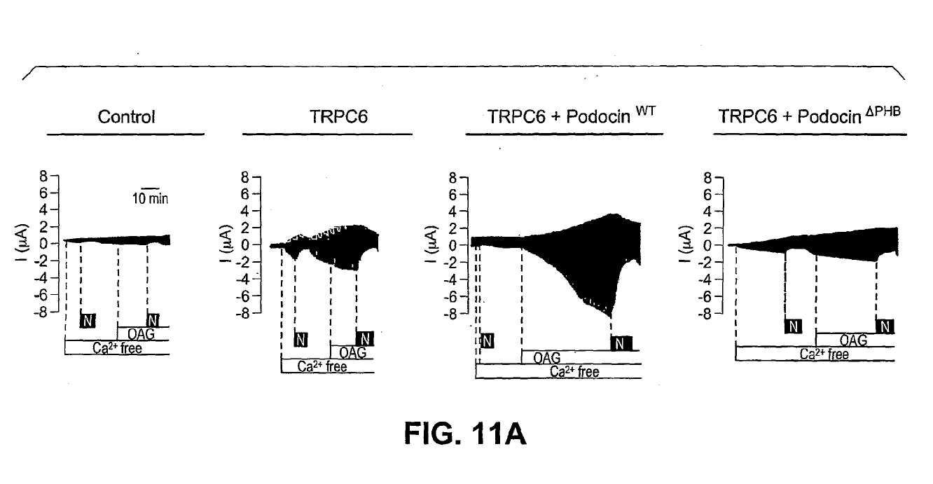

It has been found that Podocin, like MEC-2, is associated with ion channel subunits at the glomerular slit diaphragm of the kidney. As described above, mutations in the genes encoding Podocin and TRPC6 cause disruption of the kidney filter and focal segmental glomerulosclerosis [Reiser et al. (2005) Nat. Genet. 37:739-744; Winn et al. (2005) Science 308:1801-1804]. To test whether these proteins may functionally interact, tagged versions of the proteins in HEK 293T cells were co-expressed and tested for co-immunoprecipitation. Podocin coprecipitated with TRPC6 whereas a control protein did not (FIG. 9A). Similar to MEC-2, which does not influence targeting of the DEG/ENaC ion channel complex [Zhang et al. (2004) Curr. Biol. 14:1888-96], Podocin did not affect TRPC6 localization to the plasma membrane. Podocytes express TRPC6 as well as several related TRPC channels (TRPC1, 3, 4) (FIG. 10). In these experiments, PCR products were generated to indicate the expression of various TRP channels in human podocytes. A cDNA library derived from the differentiated human podocytes was used to check for expression TRPC ion channels by PCR. Primer pairs specific for the individual TRPC channels were derived from Primerbank (pga.mgh.Harvard.edu/primerbank). Consistent with the hypothesis that TRPC channels are heteromeric [Freichel et al. (2005) J. Physiol. 567:59-66], Podocin coprecipitated with these other TRPC channels but not with a control protein (FIG. 10). In these experiments, FLAG-tagged Podocin (F.Podocin^{wt}) or a control protein (F.GFP) were co-expressed with HA-tagged TRPC channels (mouse TRPC1 to TRPC6). Co-precipitating TRP channels were detected with anti-HA antibody.

Consistent with a previous study [Reiser et al. (2005) Nat. Genet. 37:739-44], immunofluorescence staining of rat kidney sections confirmed expression of TRPC6 in glomerular podocytes. Immunogold electron microscopy was used in localization experiments in which rat kidneys were perfused with ice-cold PBS, fixed in situ, and subjected to immunogold electron microscopy. TRPC6 was localized to the insertion site of the glomerular slit diaphragm (FIG. 9B), the structure that expresses Podocin [Roselli et al. (2002) Am. J. Pathol. 160:131-9]. Although TRPC6 could be detected in various compartments of the podocyte, immunoreactivity in the secondary processes of the podocyte was clearly confined to the insertion site of the slit diaphragm. Thus, Podocin colocalizes with TRPC6 in vivo. This method can be adapted to determine whether a test compound can modulate an activity of a PHB domain protein, e.g., Podocin. In such experiments, a test compound is introduced into the animal prior to sacrifice to remove the tissue being examined (e.g., kidney), or is introduced into a culture containing the organ or tissue. The localization of the PHB domain protein in the sample contacted with the test compound is compared to an untreated control. A test compound that affects the localization of the PHB domain protein is useful for modulating the protein activity. In certain cases, the localization of a second protein is also assayed (e.g., TRPC6) and a difference in the relative localization of the PHB domain protein and the second protein in the sample contacted with the test compound indicates that the test compound can modulate activity of the PHB domain protein and/or the second protein.

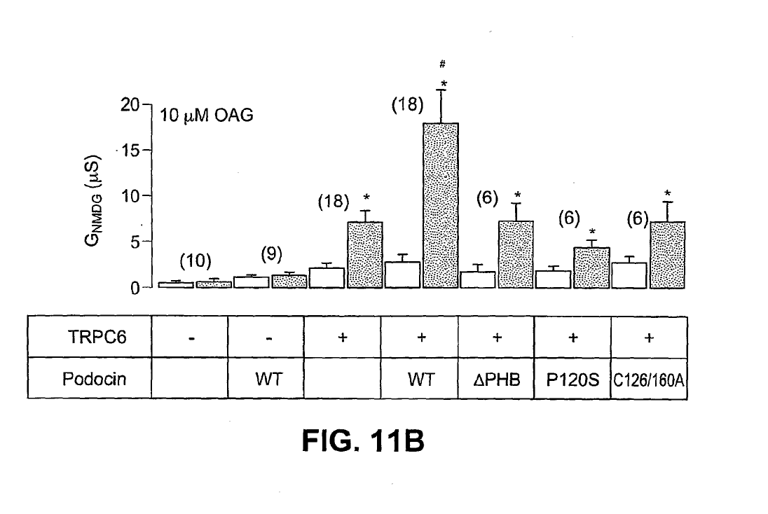

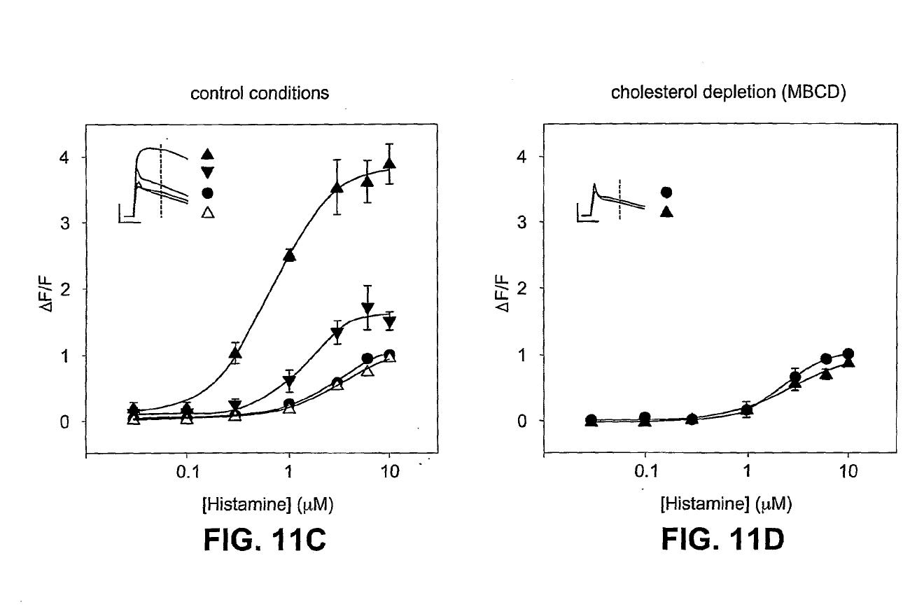

The question was examined as to whether Podocin affects TRPC6 channel activity. This was accomplished by examining TRPC6 currents in Xenopus laevis oocytes in the presence or absence of Podocin. Expression of TRPC6 induced an inward Na⁺ current in a Ca²⁺ free bath solution that was further augmented by stimulation with the membrane permeable diacylglycerol homologue 1-oleoyl-2-acetyl-sn-glycerol (OAG, FIG. 11A). This increase required the TRPC6 channel; e.g., it was not seen in water injected oocytes. Podocin, but not Podocin^{ΔPHB}, enhanced TRPC6 currents in Xenopus oocytes stimulated with 10 μM 1-oleoyl-2-acetyl-sn-glycerol (OAG). Expression of TRPC6 induced an inward Na⁺ current in a Ca²⁺ free bath solution that was further augmented by stimulation with OAG. The OAG-induced currents were significantly augmented in oocytes coexpressing TRPC6 and Podocin, but were not increased in oocytes coexpressing TRPC6 and Podocin^{ΔPHB} (FIG. 11A). These data demonstrate that Podocin interacts with TRPC6 to regulate TRPC6 activity. Therefore, compounds that modulate Podocin interaction with TRPC6 can be used to modulate TRPC6 activity.

To test whether the Podocin-mediated activation of TRPC6 also involves cholesterol binding, a mutant Podocin that was defective in cholesterol binding was coexpressed with TRPC6 in oocytes. The effect of Podocin on the TRPC6 channel currents was quantified by replacing Na⁺ in the extracellular bath solution with impermeable n-methyl-D-glucamine (NMDG) and calculating the NMDG-sensitive conductance (G_{NMDG}, FIG. 11B). Mutation of the proline residue (Podocin^{P1205}) equivalent to MEC-2(134S) or of the palmitoylation sites (Podocin^{C126/160A}) both resulted in the loss of the OAG-stimulated currents. Podocin^{P120S} did not bind cholesterol and Podocin^{C126/160A} showed weak cholesterol binding activity, but both interacted with TRPC6. Thus, Podocin increased the effect of OAG (10 μM1-oleoyl-2-acetyl-sn-glycerol; black bars in FIG. 11B) on NMDG-sensitive conductance (G_{NMDG}) of TRPC6 channels in Xenopus oocytes, but mutant Podocins did not.

These data indicate that the regulation of TRPC6 by Podocin requires cholesterol binding. Although a demonstration of an abrogation of the stimulatory activity of Podocin on TRPC6 currents would be useful to support this finding, cholesterol cannot be efficiently removed from oocytes. Therefore, instead, the effects of Podocin on the histamine-stimulated and TRPC channel-dependent increase of calcium in HeLa cells [Shirokova et al. (2005) J. Biol. Chem. 280: 11807-15], which allow efficient cholesterol depletion (FIG. 11C and FIG. 11D) was examined. Expression of Podocin resulted in a strong increase of the maximal effect to histamine stimulation on transmembrane Ca²⁺ influx (FIG. 11C). This increase was not found in cells expressing Podocin^{ΔPHB} and was strongly attenuated in cells expressing Podocin with mutated palmitoylation sites (FIG. 11C). The weaker effect of the palmitoylation site mutations mirrors that seen with the similar MEC-2 mutant in C. elegans. Consistent with a critical role for Podocin in binding and recruiting cholesterol, limited cholesterol depletion with methyl-β-cyclodextrine abolished the Podocin-dependent stimulation of Ca²⁺ influx (FIG. 11D). Although treatment of cells with methyl-β-cyclodextrine may have a variety of effects, these data together with the oocyte experiments suggest that Podocin-mediated cholesterol recruitment is essential for modulating TRPC channel function.

### Example 6

### Pannexin

It has been observed that humans whose red blood cells (RBCs) lack stomatin have hereditary stomatocytosis. RBCs swell and lyse and cannot control Na⁺ and K⁺ permeability. [Stewart (1997) Int. J. Biochem. Cell Biol. 29:271-274]. RBCs have pannexins (vertebrate proteins similar to invertebrate innexins, their gap junction proteins) ([Barbe et al. (2006) Physiology (Bethesda) 21:103-114; Locovei et al., (2006) Proc. Natl. Acad. Sci. USA 10:7655-7659]. The pannexins lead to the production of gap junctions and hemijunctions that have high conductance (comiexin 46 can also do this) (Bao et al. (2004) FEBS Lett. 572:65-68 and Locovai et al. (2006) Proc. Natl. Acad. Sci. USA 103:7655-7659].

In C. elegans, mutations of unc-79 and unc-80 result in animals that are hypersensitive to volatile anesthetics like halothane. Mutations in unc-1 (a stomatin gene), and unc-7 and unc-9 (two innexin genes) suppress this hypersensitivity, but not the sensitivity to volatile anesthetics (Morgan et al. (1990) Proc. Natl. Acad. Sci. USA 87: 2965-2969). Mutations in genes that affect cholesterol sulfation suppress unc-1 (Carroll et al. (2006) J. Biol. Chem. September 13, Epub ahead of print] and cholesterol sulfate protects RBCs from lysis [Strott and Higashi (2003) J. Lipid Res. 44:1268-1278].

Based on the finding reported herein and information derived from the art, innexins (pannexins) can bind PHB domain proteins and are regulated by them. In addition, at least certain PHB domain proteins can utilize a cholesterol related compound, cholesterol sulfate. Furthermore, the pannexins in RBCs are determined to be “fail-safe” channels that allow the cells to cope with changes in osmolarity (e.g., they are analogous to bacterial MscL channels [Perozo and Rees (2003) Curr. Opin. Struct. Biol. 13:432-442]—these open and let out solutes just before cells lyse, thus ameliorating lysis). Sequence and structural comparisons are performed using methods and programs available in the art to confirm structural or sequence similarities of innexins and pannexins with PBH domain proteins.

Experiments are conducted using methods described herein and methods known in the art to confirm that innexins and pannexins localize to the low density fraction (LDF). In addition, the binding of UNC-1 to UNC-7 and UNC-9 is confirmed using such methods known in the art. Further, binding of pannexins to stomatin is confirmed and the binding of UNC-1 and stomatin to cholesterol is confirmed using methods known in the art.

RBCs are examined to determine the parameters of a fail-safe reaction that they may have, e.g., the parameters of any release of cellular contents such as ions prior to lysis. Additional experiments are carried out to alter stomatin and pannexin content of reticulocytes, e.g., using RNAi. Cells with such altered content are then tested to determine the function of these components.

In yet another line of experiments, the effect of cholesterol sulfate on C. elegans is determined using methods disclosed herein and methods known in the art.

Compounds are tested for their ability to interact with the PHB domain protein(s) of RBCs. Such compounds are candidates for ameliorating certain effects such as lysis. For example, such compounds are candidate compounds for ameliorating conditions that are characterized by undesirable lysis of RBCs (e.g., malaria).

### Example 7

### Modulation of Amyloid Precursor Protein (APP)

Background. APP (amyloid precursor protein) can be processed to produce a 40 amino acid fragment or a 42 amino acid fragment (Aβ), the latter being a major component of amyloid plaques in Alzheimer's Disease (AD). Processing of APP appears to be different depending on the lipid environment of the protein [Ehehalt et al. (2003) J. Cell Biol. 160:113-123]. When APP is in a non-cholesterol-rich membrane environment, the 40 amino acid fragment is produced; when APP is in a cholesterol-rich environment, Aβ is produced. In the mouse cerebral cortex, the bulk of APP is found in non-cholesterol-containing membrane. A small amount of the protein fractionates with cholesterol-rich membranes, where APP has been termed “atypical lipid raft protein” [Parkin et al. (1999) Biochem. J. 344:23-30]. Specifically, the proteins remain in the DRM fraction at 37° as well as at 4°. This property is, based on the findings provided herein, expected from association with a PHB protein. A paper by Chen et al. [(2006} Biochem. Biophys. Res. Comm. 342:266-272] demonstrates that APP can bind to the PHB protein flotillin and that this binding does not depend on the PHB domain. These results indicate that the processing of APP to produce the Aβ peptide depends on binding of APP to flotillin, which changes the lipid environment of the protein, thus regulating the processing of APP.

Experiments. Experiments are conducted to determine that flotillin binds cholesterol. The procedures are as described supra for MEC-2 and Podocin. Specifically, flotillin fractionation is examined using sucrose density gradients (with and without MBCD and at 4° and 37°) confirming that it fractionates in the LDF (DRM) in an “atypical manner.” Photoaffinity cholesterol labeling of flotillin is also carried out to further confirm the association of flotillin and cholesterol.

The ability of flotillin to bind to APP and alter the properties (e.g., the cleavage pattern) of APP is determined. Sucrose density gradient fractionation and cholesterol binding of APP in HEK 293T cells are used and it is determined whether cholesterol binding of APP and/or the position of APP in such gradients is altered by coexpression of flotillin. In addition, it is determined whether the fractionation to the LDF of APP depends on the amount of flotillin expressed in the cells. Such assays are readily practiced by those skilled in the art. The binding of flotillin to APP is also confirmed. The specificity of the binding is also tested using Podocin, stomatin, and MEC-2.

Mutations in flotillin that prevent the binding of cholesterol, allow for appropriate binding to APP but not for its fractionation to the LDF.

In additional experiments mutations in APP that are associated with early-onset AD are tested for their ability to affect the fractionation and/or flotillin binding properties of APP. Such mutations are known in the art and the effect is tested using methods described herein and methods known in the art. The Alzheimer Disease and Frontotemporal Demetia Mutation Database (www.molgen.ua.ac.be/ADMutations/default.cfm?MT=1&ML=1&Page=MutByQuery& Query-tb1Contexts.ID=3&Selection=Gene %20=%20APP) lists certain mutations that are associated with early-onset AD that are clustered around the site of γ-secretase cleavage (as well as mutations in the same area that are neutral). The effects of such mutations on sucrose density gradient fractionation, cholesterol binding, and flotillin binding of the APP are determined.

The effect of flotillin on the cleavage of APP is determined, i.e., whether the presence of flotillin (and cholesterol) changes the amount of Aβ that is produced from APP. Assays for APP cleavage are well established and involve testing for the size of the specific cleaved fragments (either by gel electrophoresis methods or mass spectrometry). Such methods are incorporated into an assay using models in which flotillin is overexpressed and/or in assays in which flotillin expression is inhibited in the presence and in the absence of a compound such as cholesterol, glucocorticoid, gonadal steroid, or other compound (e.g., a test compound).

In additional experiments, the effect of steroids on APP is examined, e.g., to determine whether steroids affect the fractionation, cleavage pattern, or other features of APP (See, Green et al. (2006) J. Neurosci. 26:9047-9056). Previously, estrogen therapy was considered a means of delaying AD [Scalco and van Reekum (2006) Can. Fam. Physician 52:200-207]. Experiments are conducted to determine whether estrogen interferes with the fractionation and other properties APP in the presence or absence of flotillin. A lack of effect of estrogen or an estrogen analog indicates that the hormone is an unlikely candidate for treating AD. Other test compounds can be tested using this system as a means of identifying compounds that are candidate compounds for treating AD.

Rovelet-Lecrux et al. [(2006) Nat. Genet. 38:11-12] reported early onset AD with duplication of the APP gene. LDF localization is tested to determine if the localization is concentration dependent in transfected cells. Higher concentrations of APP may drive the binding with flotillin or some other PHB-domain protein. Such proteins can be identified using methods known in the art such as two-hybrid screening systems.

### Example 8

### Glucocorticoid Binding

To investigate whether Podocin has ligands besides cholesterol, the PHB domain of Podocin was tested for the ability to bind to the glucocorticoid, dexamethasone. In these experiments recombinantly expressed Podocin truncations were affinity purified, bound to beads and incubated with various radioactively labeled steroids. After extensive washing the amount of steroid bound to the recombinant Podocin protein was assessed by liquid scintillation counting of the beads.

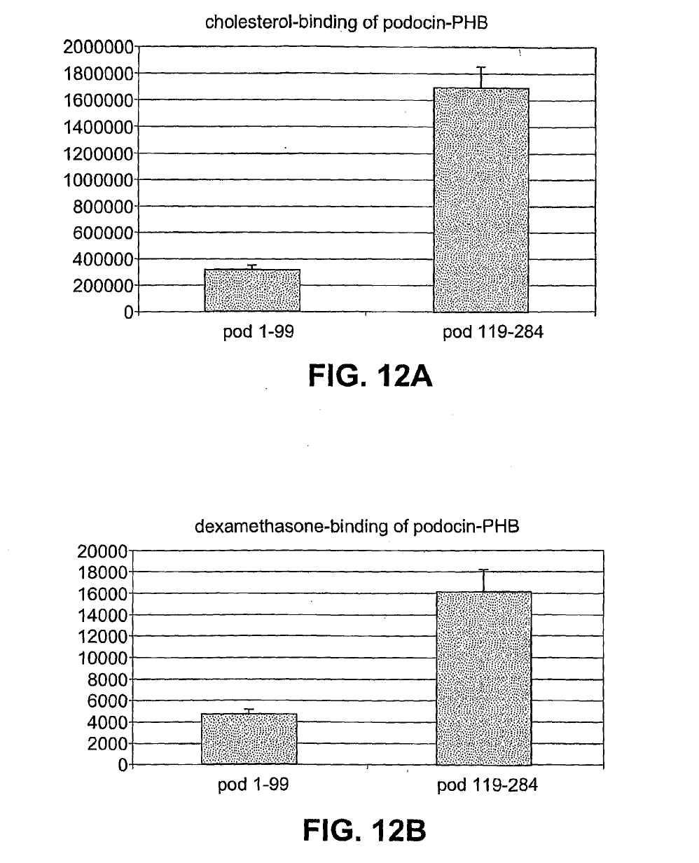

The results of these experiments demonstrate that the PHB domain of Podocin binds a glucocorticoid approximately as well as cholesterol (FIG. 12). Thus, steroids (e.g., glucocorticoids) can be suitable compounds for modulating (increasing or decreasing) activity of a PHB domain protein. These experiments also demonstrate a method of determining whether a compound can bind a PHB domain polypeptide.

Taken together, the data provided herein demonstrate that PHB domain proteins, e.g., Podocin and MEC-2, can recruit cholesterol to regulate ion channel function and that the PHB domain is required for the recruitment of sterols to membrane protein complexes. This is achieved by the combination of direct lipid interaction (via both the membrane-associated hydrophobic sequence and covalently attached palmitate chains) and multimerization by protein-protein interactions. Previous studies found that PHB domain proteins associate with cholesterol-rich fractions and suggested that the PHB proteins are components of membrane microdomains or lipid rafts. The view that cellular membranes contain lateral assemblies of lipids, with different biophysical properties to the bulk membrane, has generated great interest as a theory to explain diverse lipid-based phenomena in eukaryotic cells from yeast to man [Morrow and Parton (2005) Traffic 6:725-740]. However, recently the concept that self-organization of membrane lipids leads to the recruitment of specific proteins in lipid rafts has been challenged by a study demonstrating that in T cells such organization is not a result of the biophysical properties of lipids but solely arises by protein-protein interactions. The data provided herein address this controversy and indicate that both protein-protein and protein-lipid interactions are required for the formation of cholesterol-containing membrane protein supercomplexes that concentrate or exclude cell surface proteins. The interaction of the PHB domain proteins with specific target proteins results in an alteration of the lipid environment of these targets. Since the PHB domain is highly conserved and carried by a diverse array of proteins including stomatin, Podocin, prohibitin, lower eukaryotic proteins such as the Dictyostelium proteins vacuolin A and B and several C. elegans proteins, the present findings indicate that that PHB-protein-mediated lipid organization and recruitment into membrane protein complexes is of general importance. As the SMART database currently returns over 700 non-redundant sequences containing a PHB domain, it is clear that the finding that PHB domain proteins function as lipid organizing proteins indicates that the eukaryotic PHB domain protein family is the largest family of sterol binding proteins identified so far. The presence of this domain in some prokaryotic proteins indicates that prokaryotic PHB domain proteins may constitute primordial lipid recognition proteins that co-developed with a compartmentalized membrane composition in evolution. Accordingly, compounds that modulate prokaryotic PHB domain proteins can be useful for ameliorating undesirable effects of prokaryotes, e.g., by treating or preventing infection by a prokaryote.

This study highlights a critical role for plasma membrane cholesterol in modulating ion channel activity. Lipids have been previously implicated in regulating ion channel gating. Lipids are intimately involved in gating mechanosensitive channels in bacteria, and Kung [(2005) Nature 436:647-654] has suggested a similar role in the gating of eukaryotic channels. Without committing to any particular theory, the present findings indicate that some mechanosensitive channels require a sterol-rich microenvironment to efficiently transduce mechanical stimuli. It is shown herein that Podocin interacts with and regulates the activity of TRPC6. Mutation of TRPC6, like Podocin, causes focal segmental glomerulosclerosis, a severe kidney disorder in humans. Although the TRPC6-associated disease displays a later onset of kidney failure and milder disease than the Podocin disease, the similarity of the defects supports our concept that Podocin modulates TRPC6 function. An intriguing speculation is that Podocin, like MEC-2, may be part of a mechanosensitive protein complex at the kidney filtration barrier. TRPC1 is a component of the vertebrate stretch-activated cation channel, which is gated by tension developed in the lipid bilayer. Intriguingly, both TRPC1 and stomatin (another PHB domain protein) are both found in the “lipid raft” fraction from platelets. In analogy with Podocin and TRPC1, stomatin is a protein that should interact and regulate TRPC1.

PHB proteins also regulate signal transduction at the plasma membrane that does not involve ion channels. The present demonstration that PHB proteins recruit cholesterol to protein complexes suggests that this recruitment may be a key component of their regulatory effects. The ability of PHB domain proteins to bind cholesterol and probably other sterols could also permit them to respond dynamically to and mediate sterol action at the plasma membrane. This assigns a novel role to membrane cholesterol in facilitating the formation of membrane protein supercomplexes to regulate signal transduction in vivo. Accordingly, PHB domain proteins provide a target for regulating cell activities that engage these proteins.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for identifying a compound that modulates a PHB domain protein, the method comprising: (a) providing a polypeptide containing a PHB domain; (b) contacting the polypeptide with a compound; and (c) determining whether or not the compound binds to the polypeptide, wherein binding of the compound to the polypeptide indicates that the compound modulates the PHB domain protein.

2. The method of claim 1, wherein the polypeptide containing a PHB domain is located within a membrane.

3. The method of claim 1, wherein the method further comprises detecting whether there has been an increase or a decrease in PHB domain polypeptide activity.

4. The method of claim 4, wherein the detecting comprises (i) measuring cholesterol or sterol bound by the PHB domain, (ii) measuring protein bound by the PHB domain polypeptide, (iii) measuring multimerization of the PHB polypeptides, (iv) measuring ion channel activity, (v) measuring cholesterol or sterol recruitment, (vi) measuring enzymatic activity, or (vii) any combination thereof.

5. The method of claim 1, wherein the PHB domain polypeptide comprises: (i) a peptide consisting of a PHB domain, (ii) a peptide having a hydrophobic domain linked to a PHB domain, (iii) a peptide having a PHB domain linked to about five additional amino acids, or (iv) any of the forgoing having a mutation in a palmitoylation site.

6. The method of claim 1, wherein the PHB domain polypeptide is the PHB domain of the amino acid sequence corresponding to GenBank Accession No. AY050309.

7. The method of claim 1, wherein the PHB domain polypeptide comprises (i) the consecutive amino acids from 124 to 285 of a murine Podocin amino acid sequence, (ii) the consecutive amino acids from 138 to 300 of a C. elegans MEC-2 amino acid sequence, or (iii) a homolog or ortholog thereof.

8. The method of claim 1, wherein the compound is a cholesterol, a sterol, a steroid, a phosphatidyl ethanolamine, or an analog of any thereof.

9. The method of claim 1, wherein the method further comprises providing a known ligand of the PHB domain polypeptide in step (b) and determining the relative binding of the test compound compared to the known ligand.

10. The method of claim 1, wherein the PHB domain polypeptide is expressed in a tissue and the detecting is performed in the tissue sample.

11. The method of claim 1, wherein the PHB domain polypeptide is expressed in a cell and the detecting is performed by assaying the amount of test compound in the low density fraction (LDF) fraction derived from the cell, wherein a decrease in the amount of PHB domain polypeptide in the LDF fraction compared to a control indicates that the test compound can bind to the PHB domain polypeptide.

12. The method of claim 1, wherein the detecting comprises determining the amount of compound bound to the PHB domain polypeptide.

13. A method for identifying a compound that modulates a PHB domain protein, the method comprising: (a) providing a protein containing a PHB domain, and a target protein, wherein the target protein is capable of being bound by the PHB domain protein; (b) contacting the proteins of (a) with a compound, and (c) determining whether the compound inhibits or enhances binding of the PHB domain protein with the target protein.

14. The method of claim 13, wherein the PHB domain protein and target protein are admixed with membrane lipids.

15. The method of claim 13, wherein the method further comprises determining the affinity of the compound for the PHB domain polypeptide.

16. The method of claim 13, wherein the PHB domain protein comprises Podocin and the target protein comprises TRPC6.

17. The method of claim 13, wherein the PHB domain protein comprises stomatin and the target protein comprises TRPC1.

18. The method of claim 13, wherein the PHB domain protein comprises flotillin and the target protein comprises an Alzheimer's precursor protein (APP).

19. The method of claim 13, wherein the PHB domain protein comprises a pannexin.

20. The method of claim 13, wherein the PHB domain protein contains a mutation.

21. The method of claim 20, wherein the PHB domain protein contains a mutation in a palmitoylation site.

22. A method for identifying a compound that modulates activity of a PHB domain polypeptide, the method comprising: (a) providing a PHB domain polypeptide; (b) contacting the PHB domain polypeptide with a compound under conditions suitable for detecting PBH domain protein activity; and (c) detecting PHB domain protein activity and comparing the activity detected with activity detected from a PHB domain polypeptide in the absence of the compound, so as to identify a compound that modulates PHB domain polypeptide activity.

23. The method of claim 22, wherein the PBH domain polypeptide activity detected is cholesterol or sterol binding.

24. The method of claim 22, wherein the PHB domain polypeptide activity detected is ion channel activity.

25. The method of claim 24, wherein the PHB domain polypeptide is the amino acid sequence corresponding to GenBank Accession No. AY050309 and the ion channel activity detected is a TRPC ion channel.

26. The method of claim 22, wherein the PHB domain polypeptide is a PHB domain protein.

27. The method of claim 22, wherein the PHB domain polypeptide is the amino acid sequence corresponding to GenBank Accession No. AY050309.

28. The method of claim 22, wherein the PHB domain polypeptide comprises (i) the consecutive amino acids from 124 to 285 of a murine Podocin amino acid sequence, (ii) the consecutive amino acids from 138 to 300 of a C. elegans MEC-2 amino acid sequence, or (iii) a homolog or ortholog thereof.

29. The method of claim 22, wherein the compound is a cholesterol, a sterol, a steroid, a phosphatidyl ethanolamine, or an analog of any thereof.

30. The method of claim 1 or claim 22, wherein the PHB domain polypeptide is in an intact organism or tissue from an organism.

31. The method of claim 30, wherein the organism is a C. elegans, and the assay is a touch sensitivity assay, wherein a test compound that binds to MEC-2, interferes with binding of MEC-2 to cholesterol, interferes with binding of MEC-2 to other PHB domain proteins, interferes with the binding of MEC-2 to MEC-2, to cholesterol or to themselves or other PHB-domain proteins modulates touch sensitivity in a wild-type C. elegans by decreasing touch sensitivity, or in a mutant for touch sensitivity by increasing touch sensitivity.

32. The method of claim 30, wherein the organism is a C. elegans, and the assay is a touch sensitivity assay, wherein a test compound that binds to a PHB domain protein interferes with the binding of a PHB domain protein to cholesterol, interferes with the binding of the PHB domain protein to itself, interferes with the binding of the PHB domain protein to a different PHB domain protein modulates touch sensitivity in a wild-type C. elegans by decreasing touch sensitivity, or in a mutant for touch sensitivity, by increasing touch sensitivity.

33. The method of claim 22, wherein the detecting comprises detecting ion channel activity.

34. The method of claim 33, wherein the detecting of ion channel activity is performed using a X. laevis oocyte system.

35. The method of claim 34, wherein the detected ion channel activity is at least one of Na⁺ channel activity, Ca²⁺ channel activity, K⁺ channel activity, or non-selective cation channel activity.

36. The method of claim 22, wherein the detecting comprises detecting membrane receptor activity or membrane protein activity.

37. The method of claim 36, wherein membrane receptor activity is a G-protein coupled membrane receptor activity or a hormone receptor activity.

38. The method of claim 22, wherein the compound can bind to a pannexin and can modulate lysis in a red blood cell.

39. The method of claim 22, wherein the PBH domain polypeptide is a mutated PHB domain polypeptide.

40. The method of claim 39, wherein the PHB domain polypeptide has a mutation in at least one predicted palmitoylation site.

41. A method of identifying a compound that can modulate multimerization of a PHB domain polypeptide, the method comprising: (a) providing a population of PHB domain polypeptides; (b) contacting the population with a compound; and (c) determining whether the PHB domain proteins form multimers, wherein an inhibition of multimerization in the population of (b) as compared to a population in the absence of a compound, indicates that the compound can modulate multimerization of a PHB domain polypeptide.

42. The method of claim 41, wherein the compound is a cholesterol, sterol, steroid, phosphatidylethanolamine or a compound related to any of the forgoing.

43. An isolated peptide consisting essentially of a PHB domain.

44. An isolated peptide consisting essentially of a PHB domain linked to 5 additional amino acids.

45. The peptide of claim 43 or 44, wherein the isolated peptide has the amino acid sequence of amino acids 124-285 of SEQ ID NO:1 or amino acids 138-300 of SEQ ID NO:2.

46. A composition comprising a peptide of claim 43, 44 or 45 and a pharmaceutically acceptable carrier.

47. The composition of claim 43 or 44, wherein the PHB domain is a MEC-2 PHB domain, a Podocin PHB domain, an MEC-2 PHB domain having 5 additional amino acids at the amino terminus, or a Podocin PHB domain having 5 additional amino acids at the amino terminus.

48. A method for modulating activity of a PHB domain polypeptide, the method comprising contacting a cell expressing the PHB domain polypeptide with an agent that can bind to the PHB domain of the PHB domain polypeptide.

49. The method of claim 48, wherein the agent is an antibody that specifically binds a PHB domain protein.

50. The method of claim 48, wherein the agent is a peptide consisting essentially of a PHB domain.

51. The method of claim 48, wherein the agent is a cholesterol, a sterol, or a steroid.

52. The method of claim 48, wherein the agent is lathosterol, ergosterol, or 7-dehydro cholesterol.
